(19) United States
(12) Patent Application Publication
(10) Pub. No.: US 2010/0184037 A1
(43) Pub. Date: Jul. 22, 2010

(54) DEATH ASSOCIATED PROTEIN KINASE 1 (DAPK1) AND USES THEREOF FOR THE TREATMENT OF CHRONIC LYMPOCYTIC LEUKEMIA

(75) Inventors: Christoph Plass, Columbus, OH (US); Albert de la Chapelle, Delaware, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/529,214

(22) PCT Filed: Feb. 28, 2008
(86) PCT No.: PCT/US08/02625

§ 371 Date: Mar. 2, 2010

Related U.S. Application Data
(60) Provisional application No. 60/904,549, filed on Mar. 2, 2007.

(51) Int. Cl. C12Q 1/68; C12N 9/12; C07H 21/04

(52) U.S. Cl. 435/6; 536/23.5; 435/194; 536/24.1; 536/23.1

(57) ABSTRACT

A method for determining susceptibility to chronic lymphocytic leukemia in a subject includes determining a loss or reduced expression of death associated protein kinase 1 (DAPK1) or fragments or functional equivalents thereof.

### CROSS-REFERENCE TO RELATED APPLICATIONS

This application claims the benefit of U.S. Provisional Application No. 60/904,549, filed Mar. 2, 2007, the disclosure of which is incorporated herein by reference.

### STATEMENT REGARDING FEDERALLY SPONSORED RESEARCH

This invention was made with Government support and the Government has rights in this invention under one or more of the following grants: T32 CA106196 fellowship in Cancer Genetics. This publication was supported by National Cancer Institute grants CA110496, CA81534 to the CLL Research Consortium, P30 CA16058, and by NIH grant 5U01 CA86389.

### TECHNICAL FIELD AND INDUSTRIAL APPLICABILITY OF THE INVENTION

This invention is directed to certain novel compounds, methods for producing them and methods for treating or ameliorating a kinase mediated disorder, and in particular to death associated protein kinases 1 (DAPK1). More particularly, this invention is directed to compounds useful as selective kinase inhibitors, methods for producing such compounds and methods for treating or ameliorating chronic lymphocytic leukemia (CLL).

### BACKGROUND OF THE INVENTION

Chronic lymphocytic leukemia (CLL) is one of the most common chronic leukemias in the Western world. There were 8,190 new cases of CLL in the United States in 2004 (Kasper and Harrison, 2005). Because of the long average survival, the prevalence is much higher. CLL is mostly a disease of the elderly and is more common in males than in females. CLL is known to sometimes have a protracted, almost asymptomatic course over 10-20 years. In other cases, progression can rapidly lead to death within months or a few years. There is, however, no cure for CLL (Kasper and Harrison, 2005). While CLL has a B1 B-cell immunophenotype (CD5+, CD19+, CD20+, CD23+), the normal non-malignant counterpart of this leukemia has not been definitely demonstrated. To date, environmental factors conveying increased risk have not been convincingly demonstrated.

Familial occurrence of CLL has been noted in up to 10% of cases (Yuille et al., 2000), however large pedigrees with many affected individuals are exceedingly rare. Thus, most familial agglomerations consist of 2 or 3 affected first- or second-degree relatives (Sellick et al., 2005), even with the inclusion of other lymphoid malignancies (Yuille et al., 2000). Large case-control studies concluded that the risk ratio (RR), a measurement for the frequency of the disease in first-degree relatives of CLL probands, was higher for CLL (or CLL and other Non-Hodgkin's lymphoid malignancies studied together) than for most other cancers (Goldgar et al., 1994). While the average RR for all cancers in a US study was approximately 2.1, CLL showed a RR of 5.0, the fourth highest of all cancers (Goldgar et al., 1994; Risch, 2001). For CLL, a RR of 7.5 was recently calculated in Sweden (Goldin et al. 2004). In a study of the entire Icelandic population, lymphoid leukemia had the second highest RR of all malignancies studied (Amundadottir et al., 2004).

This evidence of high heritability has prompted investigators to collect samples from CLL families to conduct genome-wide searches for linkage. The findings, in particular from one US and one European-led consortium (Goldin et al., 2003; Sellick et al., 2005) have been limited due to weak evidence for linkage and occurrence at many loci that were different between the two studies. Two loci in chromosome bands 11pi i and 13g21 were backed by statistically significant evidence, but no gene has been implicated in studies to date.

Genetic as well as epigenetic aberrations are intricately connected in the neoplastic process, and both therefore need to be considered in order to understand the molecular mechanisms underlying malignant transformation. Studies uncovering epigenetic aberrations in CLL have accelerated the search for CLL related genes. A genome-wide DNA methylation analysis of CLL samples, with both abnormal and normal karyotype, identified almost 200 novel genes that are epigenetically silenced in CLL. This study concluded that on average 4.8% of all CpG islands in a CLL genome could be targeted for aberrant DNA methylation and associated gene silencing (Rush et al., 2004). The role of aberrant methylation in CLL was highlighted by the finding of promoter methylation directed gene silencing of ZAP-70 and TWIST2, preferentially in subgroups of CLL defined by genetic alterations (Corcoran et al., 2005; Raval et al., 2005). Furthermore, secreted frizzled-related proteins, negative regulators of the Wnt signaling pathway, which controls normal apoptotic behavior and B-cell development, were frequently methylated and transcriptionally silenced in CLL (Liu et al., 2006). The picture that is emerging from these findings is that promoter methylation contributes significantly to global expression changes that have been described for CLL (Rosenwald et al., 2003; Wiestner et al., 2003).

Therefore, there is a need to inhibit protein kinases, particularly death associated protein kinase 1, for treatment of human diseases.

Considering the above-mentioned, there is a need for therapeutic strategies to treat chronic lymphocytic leukemia.

### SUMMARY OF THE INVENTION

In one aspect, there is provided a method for determining susceptibility to chronic lymphocytic leukemia in a subject includes determining a loss or reduced expression of death associated protein kinase 1 (DAPK1). In certain embodiments, the lost or reduced expression due to epigenetic silencing of DAPKI by promoter methylation.

In another aspect, there is provided a method for determining susceptibility to chronic lymphocytic leukemia in a subject by determining the occurrence of reduced DAPK1 expression in combination with frequent promoter methylation in the CLL cells. The DAPK1 silencing is due to modulation of upstream signal, where at least one signal comprises HOXB7, a homeobox containing transcription factor mediating a variety of developmental processes, including hematopoietic differentiation and lymphoid development.

In another aspect, there is provided a composition that includes a single-nucleotide germline mutation (c.1-6531A>G) [SEQ ID NO: 1] upstream of DAPK1, which segregates with a CLL phenotype. Also, a tumor suppressor for chronic lymphocytic leukemia (CLL) includes one or more epigenetic silencing and/or mutations in death associated protein kinase 1 (DAPK1).

In yet another aspect, there is provided a composition that includes at least one mutation in the homeobox containing transcription factor binding site for HOXB7, which is a binding site in DAPK1 regulatory region and/or promoter methylation which results in significant reduction in the expression of the pro-apoptotic gene DAPKI in familial cases of CLL and in sporadic CLL.

In still another aspect, there is provided a DAPKI promoter construct #2 (c.1-1545 to c.1-1151bp) [SEQ ID NO: 2], covering bisulfite region A1 (displaying extensive promoter methylation). Also provided is a CLL specific SNP, c.1-6531A>G. Also provided is a 357 by fragment including c.1-6531A (DAP-A) [SEQ ID NO: 3] ligated upstream into luciferase construct #1 containing a DAPKI promoter. Also provided is a 357 by fragment including c.16531G (DAP-G) [SEQ ID NO: 4] ligated upstream into luciferase construct #1 containing a DAPKI promoter.

In yet another aspect, there is provided a method for determining susceptibility to chronic lymphocytic leukemia that includes integrating genetic and epigenetic data for the discovery of predisposing genes in cancer.

Also provided is a method for determining susceptibility to chronic lymphocytic leukemia that includes combining linkage analysis and epigenetic studies using at least the down regulation of DAPK1.

One method for determining susceptibility to chronic lymphocytic leukemia includes screening of familial CLL cases for DAPKI promoter methylation. In a particular method the determination of susceptibility to chronic lymphocytic leukemia can include identifying at least one DNA methylation event that is reversible for the development of novel treatment regimens in CLL involving epigenetic therapies for gene reactivation.

Also provided is a method of diagnosing a human subject with CLL. The method can include: detecting a level of expression of a marker selected from a group of markers associated with CLL in a test sample from the human subject; and detecting the level of expression of the marker in a control sample from normal tissue from the human subject, wherein the level of expression of the marker in the control sample differs from the level of expression of the marker in the test sample when the subject is afflicted with CLL, and wherein the marker is encoded by a gene. The test sample from the subject can be cells obtained from the subject, such as for example, cells obtained from blood.

The levels of expression of the marker in the control sample and in the test sample are assessed by a method comprising: contacting a first array of probes with a first population of nucleic acids derived from one or more cells from the test sample; contacting a second array of probes with a second population of nucleic acids derived from one or more cells from the control sample; and determining relative hybridization of the first array of probes to the first population of nucleic acids relative to hybridization of the second array of probes to the second population of nucleic acids. The first and second population of nucleic acids can be RNA and/or DNA. The first population of nucleic acids can be amplified prior to contacting to the first array of probes or the second population of nucleic acids is amplified prior to contacting the second array of probes. The marker can be a nucleic acid, such as RNA or DNA. One or more nucleic acids can be amplified prior to assessing the sample.

There is also provided herein a method for monitoring the progression of CLL in a human subject that includes: detecting in a first sample obtained from the human subject at a first point in time, a level of expression of a marker selected from a group of markers associated with CLL; detecting in a subsequent sample obtained from the human subject at a subsequent point in time, the level of expression of the marker, and comparing the level of expression detected in the first and subsequent detecting samples in order to monitor the progression of CLL, wherein the marker is encoded by a gene selected as described herein. The first and the subsequent samples can be cells obtained from the subject, such as, for example, blood cells. Also, the control sample from the subject can comprise cells obtained from the subject.

In still another aspect, there is provided a method of preventing recurrence of a CLL that comprises administering an effective amount of a composition comprising an effective inhibitor of DAPK1. The method is useful as a therapeutic tool to prevent recurrence or further tumor spread.

In still another aspect, there is provided a composition for affecting CLL comprising a DAPK1 regulator.

In still another aspect, there is provided an animal model for examining CLL models that comprises administering one or more DAPK1 regulators. Also, provided is a method for inhibiting CLL cell migration comprising administering an effective amount of a composition comprising one or more DAPK1 inhibitors.

In still another aspect, there is provided a method for treating a patient in need thereof, comprising administering a therapeutically effective amount of a pharmaceutical composition which comprises a substance that regulates the activity of DAPK1, as an active ingredient.

Various objects and advantages of this invention will become apparent to those skilled in the art from the following detailed description of the preferred embodiment, when read in light of the accompanying drawings.

### BRIEF DESCRIPTION OF THE DRAWINGS

FIGS. 1A and 1B shows an analysis of DAPK1 promoter methylation by MassARRAY analysis in CLL samples:

FIG. 1A—Upper panel shows a schematic representation of the DAPK1 gene showing the location of CpG island (black bar) and the four bisulfite reactions amplicons (A1 to A4). Amplicons A1 to A4 cover 34, 39, 28 and 35 CpGs, respectively and extend from c.1-1573 to c.1-239 bps. An arrow indicates the predicted transcription start site. The lower panel shows a heat map of quantitative methylation data for the DAPK1 promoter region. Each line represents a sample and each square an analyzed CpG dinucleotide. Methylation frequencies extend from light yellow (0%) to dark blue (100%). Gray indicates no available data. The samples studied included seven CD19+ selected control B-cells, four control blood cells, seven CD19+ selected CLL cells, blood cells from 62 sporadic CLL samples, Raji and Jurkat cells. Asterisk indicates CLL sample that showed less than 10% methylation.

FIG. 1B—Plot of percentage methylation in controls (seven CD 19+ selected B-cells and four PBL samples; total=1 l), CD 19+ selected CLL cells (n=7), unselected CLL cells (n=62), Raji and Jurkat cell lines.

FIGS. 2A-2E show epigenetic silencing of DAPK1 in CLL:

FIG. 2A—pGL3 luciferase constructs ligated with different DAPK1 5′ upstream inserts (#1, #2, #3, and #4 were transfected into Jurkat cells and reporter activity was studied 48 hrs after transfection. SV40 promoter ligated to the luciferase reporter was used as a positive control (Ct+) and the pGL3 vector without insert was used as a negative control (Ct−). Luciferase activity for each construct is shown relative to Renilla expression. A schematic of DAPK1 upstream region relative to the predicted transcription start site is shown.

FIG. 2B—Quantitative DAPK1 expression analysis using SYBR green RT-PCR was performed on RNA extracted from normal B-cells and seven CD19+ selected CLL samples. The expression is shown relative to one B-cell (defined as 1.0).

FIG. 2C—DAPK1 expression in 50 CLL samples as measured by semi quantitative RT-PCR and compared to its expression in 6 normal B cells. The distribution is significantly different (p<0.01).

FIG. 2D—Luciferase assays in 293T cells with either methylated or unmethylated DAPK1construct #2. Error bars indicate SD.

FIG. 2E—Raji cells were treated with 0.5 μM Decitabine. RT-PCR was performed for DAPK1 and GAPDH on untreated and treated cell lines. cDNA synthesized from Jurkat cells was used as a positive control.

FIG. 2F—Bisulfite treatment for untreated and 6 and 12 days 5-aza-2′-deoxycytidine treated (0.5 μM) Raji cells. Bisulfite treated DNA was amplified for the BS1 region (c.1-1509 to c.1-1262) comprising 30 CpGs and cloned. Sequencing was done for 7-10 clones for each sample. Each row represents a clone. The open circles indicate unmethylated CpG, and closed circles indicate methylated CpG. The overall methylation frequency is given in (%) on the right.

FIGS. 3A-3E show DAPK1 regulates apoptosis in lymphoid cells:

FIGS. 3A and 3B—DAPK1 expression was studied in two different CLL samples (CLL1 and CLL2) cultured with and without HeLA cells for 1 to 6 days as shown. RNA was extracted and RT-PCR was performed on CLL samples. Raji and Jurkat cells were used as negative and positive controls respectively. GAPDH was used as an internal control.

FIG. 3C—A heat map of quantitative methylation data for the DAPK1 promoter region within amplicon A1 in CLL1 cells at day 0 and CLL1 cells cultured with and without HeLA cells at day 3 as measured by the MassARRAY system. Each line represents a sample and each square an analyzed CpG dinucleotide. Methylation frequencies extend from light yellow (0%) to dark blue (100%).

FIG. 3D—DAPK1 expression in Jurkat cells stably transfected with either vector alone (pRS), DAPK1 siRNA-A or DAPK1 siRNA-C as measured by Western blot. Tubulin expression served as a control.

FIG. 3E—Percent live cells were measured in Jurkat cells stably transfected with vector alone or DAPK1 siRNA-C, treated with activating Fas antibody (100 ng/ml). After 16 hrs, cells were harvested and suspended in binding buffer with annexin V-FITC and propidium iodide, followed by flow cytometry to assess cell death.

FIG. 3F—p53 expression in cells treated with either no, 50 ng/ml, or 100 ng/ml Fas-activating antibody.

FIGS. 4A-4F show DAPK1 expression in CLL family 4532:

FIG. 4A—Pedigree of CLL family #4532. Open circle and square represents unaffected female and male, respectively, while closed circle and square represents affected female and male.

FIG. 4B—Sequencing of the c.114G>A SNP from genomic DNA and eDNA of two unaffected (IV-2 and IV-4) and two affected (III-3 and III-4) individuals from the family #4532.

FIG. 4C—Semi-quantitative SYBRgreen RT-PCR on RNA isolated from monochromosomal hybrid clones with either WT or the CLL chromosome 9 from fibroblast cells of individual 111-4. RPL4 was used as an internal control.

FIG. 4D—DAPK1 expression from WT and CLL alleles in monochromosomal hybrids. Jurkat and WAC3CD5 cells were used as positive controls (Ct+) and NIH3T3 cells were used as the negative control (Ct−) to show that the antibody used is specific to human DAPK1.

FIG. 4E—Allelic expression of DAPK1 in affected and unaffected family members. RNA was extracted from one unaffected (IV-3) and one affected (III-4) fibroblast cell line and RT-PCR was performed using primers that amplify the c.1510G>A SNP. The PCR products were cloned and genotyped for either the A or the G allele.

FIG. 4F—Allelic variation in DAPK1 expression from the two alleles in diploid cells was studied using c.1608C>T SNP as a marker. The A/G ratio in cDNA was normalized to A/G ratio from the genomic DNA.

The RT-PCR product amplifying SNP c.1510A>G in unaffected (IV-3, FIG. 4E) and affected (III-4, FIG. 4F) fibroblast cell lines was cloned, and individual clones were genotyped. Shown is the percentage of A and G clones in IV-3 and III-4, and n is the number of clones studied. The difference in allelic expression of DAPK1 in III-4 and IV-3 was statistically significant (p<0.01).

FIGS. 5A-5F show mutation at c.1-6531 bp regulates DAPK1 expression in CLL family:

FIG. 5A—A 357 bp PCR product with SNP c.1-6531A>G was ligated upstream to DAPK1 promoter (c.1-2215 to c.1-1151) reporter construct with either A (DAP-A) or G (DAP-G) as SNP.

FIG. 5B—The DAPK1 promoter alone (#1), DAP-A and DAP-G constructs were transfected into Jurkat cells and the luciferase activity was measured. Renilla expression was used as transfection control.

FIG. 5C—Nuclear extracts from Jurkat cells were analyzed by EMSA assay using WT or CLL oligo. Five specific bands are marked.

FIG. 5D—For competition EMSA, where indicated, 10 or 50-fold molar excess concentrations of cold oligos relative to the radiolabelled oligo were used. Unlabeled Oct-1 oligo was used as a negative control.

FIG. 5E—EMSA assay was performed using WT mutant and CLL mutant oligo where the adjacent bases to the c.1-6531 SNP were mutated.

FIG. 5F—For the supershift assay, antibodies against HOXB7, USF2 and MSX2 were added in Jurkat nuclear extract, and gel shift was studied using CLL oligo.

FIG. 5G—DAPK1 expression in normal cells and CD19 selected CLL cells. Semi-quantitative RT-PCR was performed on four normal B-cells, three each of normal T-cells and granulocytes and seven selected CLL samples to study expression of DAPK1. GAPDH expression was used as an internal control.

FIG. 5H—HOXB7 expression in normal cells and CD19 selected CLL cells. Semi-quantitative RT-PCR was performed on four normal B-cells, three each of normal T-cells and granulocytes and seven selected CLL samples to study expression of HOXB7. GAPDH expression was used as an internal control.

FIGS. 6A-6E show down-regulation of DAPK1 expression by HOXB7:

FIGS. 6A and 6B—Fibroblast cell line from affected (III-4) and unaffected (IV-4) family members were transfected with 60 nM of HOXB7 siRNA or scrambled siRNA (Ct−) for different time points and HOXB7 expression was studied by quantitative SYBR green RT-PCR. HOXB7 expression in untreated cells was set as 1.0.

FIGS. 6C and 6D—DAPK1 expression was studied in the fibroblasts transfected with HOXB7 siRNA or scrambled siRNA (Ct−) for different time points by quantitative SYBR green RT-PCR.

FIG. 6E—Allele specific expression was studied in fibroblasts from III-4 and IV-4 before and after transfection of HOXB7 siRNA. RT-PCR was performed using primers that amplify the het c.1510G>A. The PCR products were cloned and genotyped. The plot shows the ratio between A and the G allele.

FIGS. 7A-7B show DAPK1 expression and promoter methylation in CLL cells of family #4532:

FIG. 7A—shows semi-quantitative RT-PCR for DAPK1 with GAPDH as an internal control on RNA extracted from blood cells of #4532 family members III-1, 111-2, III-3, III-4 and IV-5 and from separated CD19+ normal B-cells from 3 healthy volunteers.

FIG. 7B—DNA methylation analysis in CLL cells from affected and unaffected family members. Bisulfite treated DNA was amplified for unaffected (IV-1) and affected (III-1 and III-4) for the BS1 and BS2 regions. The PCR products were cloned and sequenced. Each row represents a clone. The open circles indicate unmethylated CpG, and closed circles indicate methylated CpG.

FIGS. 8A-8E show DAPK1 promoter methylation and histone tail modifications:

FIG. 8A—Schematic of the two regions amplified in ChIP assay, ChIP1 (c.1-1208 to c.1-1151 bp) and ChIP2 (c.1-1210 to c.1-1061 bp). DAPK1 exon 1 (black box) and the promoter region frequently methylated in CLL samples (gray line) are highlighted.

FIG. 8B—COBRA assays for two regions in the DAPK1 promoter, performed on Ramos (Burkitt Lymphoma), BJAB (Atypical Burkitt Lymphoma (EDV-), MEC-1 (CLL), MEC-2 (CLL), Was3CD5 (CLL), Daudi (Burkitt Lymphoma) 697 (ALL), RS11846 (Non-Hodgkin Lymphoma) RS 4;11 (ALL) cell line DNAs and CD19+ selected B-cells. (M) indicates digested PCR products representing the methylated portion and (UM) represents the unmethylated portion.

FIG. 8C—RT-PCR for DAPK1 and GAPDH expression in Raji, WAC3CD5 and Jurkat cell lines. cDNA isolated from RNA mixture of different tissue and B-cells were used as positive control.

FIG. 8D—Acetylation of histone H3 at lysine 9 and histone H4 are hallmarks of active chromatin. Histone H3 and H4 acetylation status was studied in Raji, WAC3CD5 and Jurkat cell lines within ChIP2 region using anti-acetylated histones H3 and H4 antibodies. No antibody or IgG was used as a negative control.

FIGS. 8E and 8EE—Quantitative ChIP assay for ChIP 1 region in Raji, Jurkat and WAC3CD5 cell lines using anti acetylated histones H3-K9 and H4 antibody. IgG was used as a negative control. The cell lines expressing DAPK1, (Jurkat and WAC3CD5) were enriched for ac-H3-K9 and ac-H4 in DAPK1 promoter region, while in Raji cells, the same region lacked the markers of open chromatin.

FIG. 9—Shows cold competition with A oligo and cold competition with G oligo at 10× and 50×.

FIG. 10—shows the sequence information for SEQ ID NOs. 1-8.

### DETAILED DESCRIPTION OF THE PREFERRED EMBODIMENT

In one aspect, there is disclosed herein that loss or reduced expression of death associated protein kinase 1 (DAPK1) underlies cases of heritable predisposition to CLL and the majority of sporadic CLL. Epigenetic silencing of DAPK1 by promoter methylation occurs in almost all sporadic CLL cases.

Furthermore, a rare, single-nucleotide germline mutation (c.1-6531A>G) upstream of DAPK1, which segregates with the CLL phenotype in a large family was detected. DAPK1 expression of the mutated allele is downregulated by 75% in germline cells due to increased HOXB7 binding. In the blood cells from affected family members, promoter methylation results in additional loss of DAPK1 expression. Thus, reduced expression of DAPK1 can act both as a germline predisposing event, and as an epigenetic or genetic somatic event causing or contributing to the CLL phenotype.

Epigenetic and genetic data were analyzed to identify a novel tumor suppressor in CLL. For the first time, we present evidence that epigenetic silencing and/or mutations in death associated protein kinase 1 (DAPK1), a positive mediator of apoptosis, contribute to familial CLL and is silenced in virtually all cases of sporadic CLL. A mutation in the homeobox containing transcription factor, HOXB7, binding site in DAPK1 regulatory region and/or promoter methylation resulted in significant reduction in the expression of the pro-apoptotic gene DAPK1 in familial cases of CLL and in sporadic CLL.

Frequent Epigenetic Inactivation of DAPK1 in CLL:

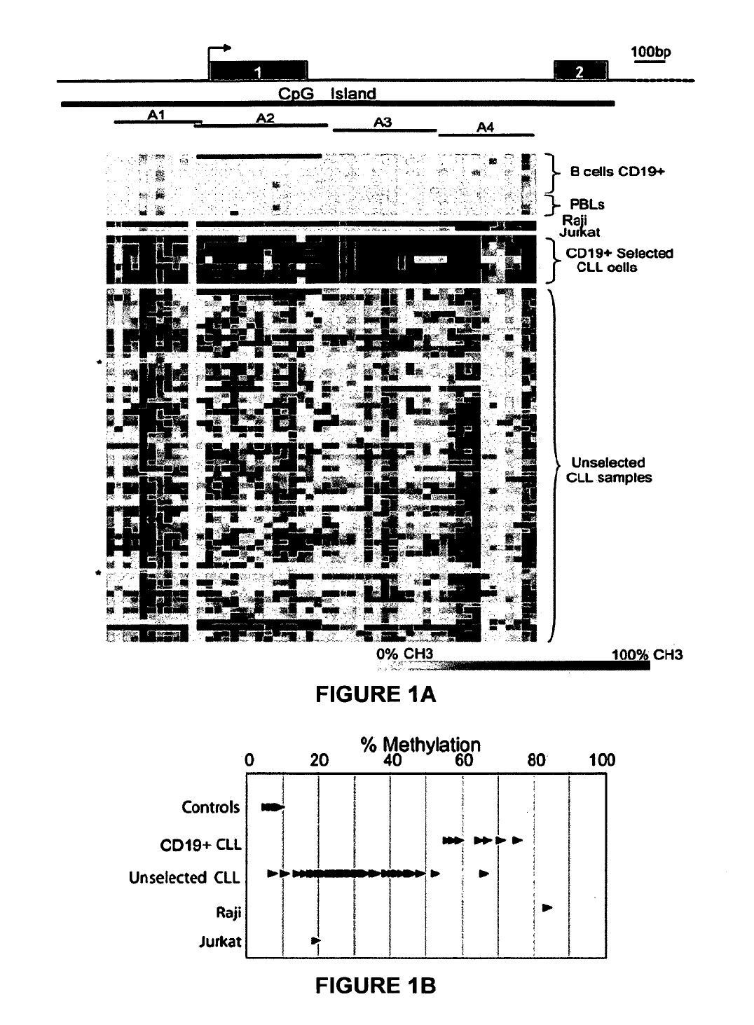

DAPK1 was initially isolated as a positive mediator of apoptosis induced by interferon gamma (INF-y) (Deiss et al., 1995). A previous study, performed by methylation-specific PCR, demonstrated low frequencies of aberrant DNA methylation of the DAPK1 promoter in CLL samples (Katzenellenbogen et al., 1999). We reevaluated and expanded the analysis of DNA methylation events in the extended DAPK1 promoter CpG island (FIG. 1A) and performed quantitative high-throughput analysis of DNA methylation using the MassARRAY system. Four bisulfite reactions, A1 to A4 (c.1-1573 to c.1-239), including 34, 39, 28 and 35 CpGs, respectively, were designed (FIG. 1A). While the average DNA methylation frequency in seven samples of normal CD19+ B-cells and four control peripheral blood cells was 7.6% or 6.3% (range 5.1% to 8.9%) respectively, we saw an average of 64% (range 55.9% to 83.9%) DNA methylation in seven CD19+ selected CLL samples (FIG. 1B).

To determine the frequency of DAPK1 promoter methylation in sporadic CLL samples, we evaluated peripheral blood cells from 62 sporadic CLL patient samples. These CLL samples showed varying levels of DAPK1 promoter methylation, ranging from 7.2% to 66.1%. Interestingly, only 2 out of 62 CLL samples showed methylation levels in the normal range (<11%) whereas all other samples showed levels >11%. The distribution of DNA methylation levels in CLL samples is significantly different from the one seen in normal cells (p<0.0001). Raji cells were methylated (83.9%) whereas Jurkat cells were unmethylated in regions A1 to A3 but methylated in A4.

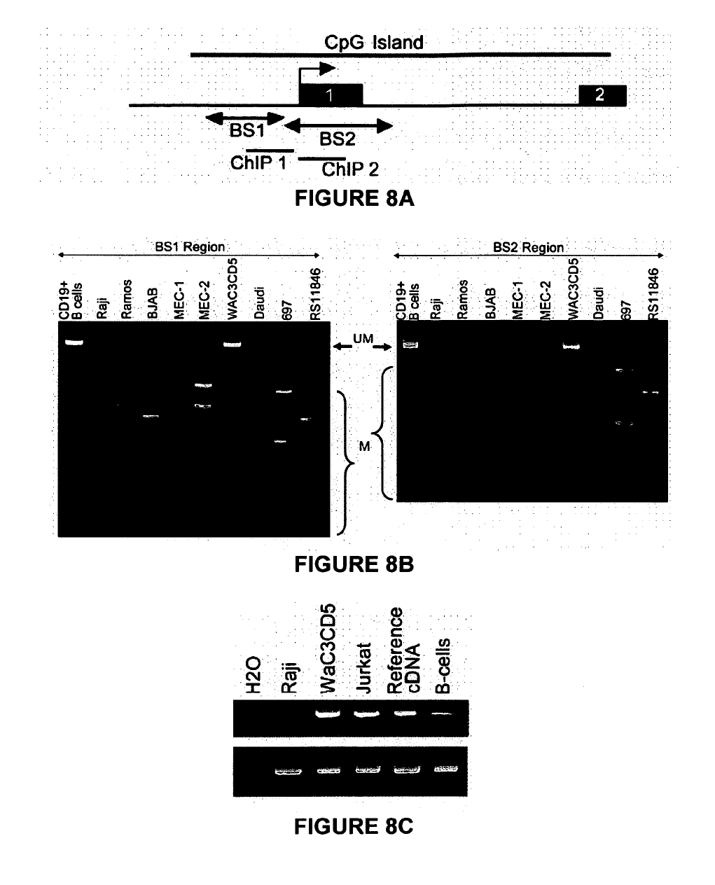

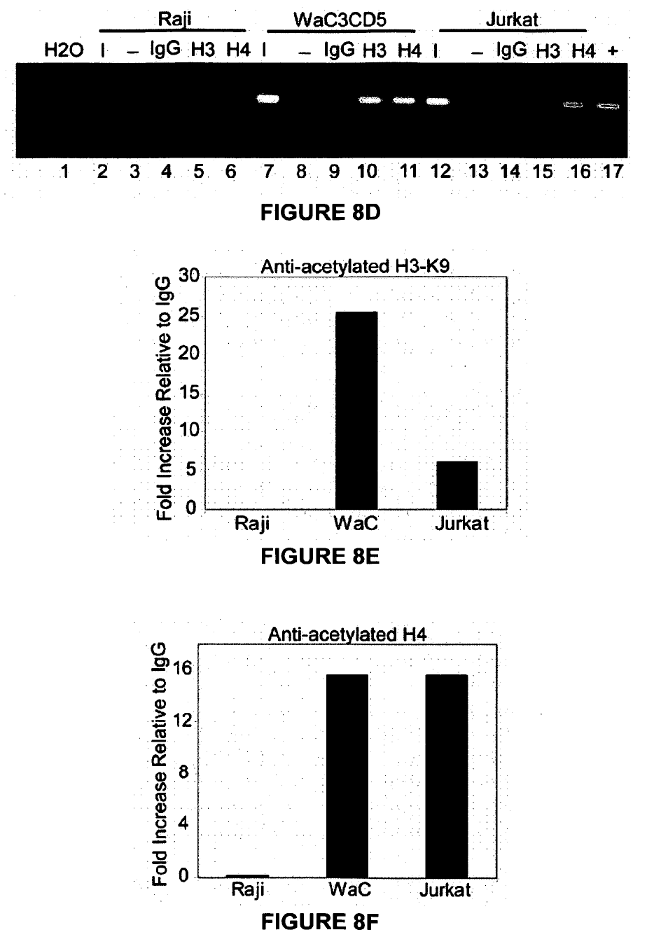

With the exception of Wac3CD5, all cell lines demonstrated aberrant DNA methylation as compared to CD19+ selected B cells (FIGS. 8A-8B). Chromatin immunoprecipitation (ChIP) assay, using anti-acetylated histone H3 and H4 antibodies, showed that in Raji cells (methylated), the DAPK1 promoter was not associated with either ac-H3 or ac-H4 histones, while WAC3CD5 and Jurkat cell lines (unmethylated) had acetylated histones within this region (FIGS. 8C-8F).

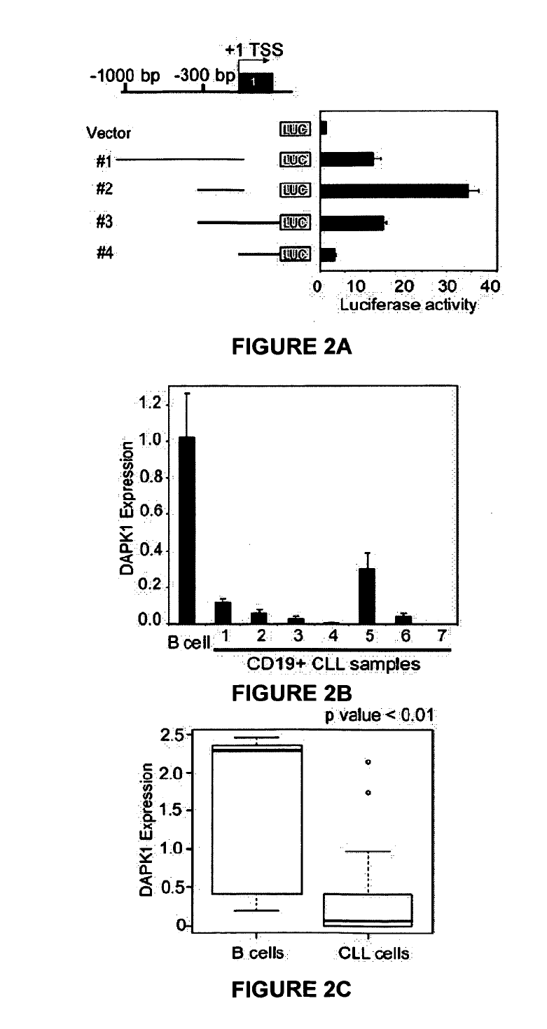

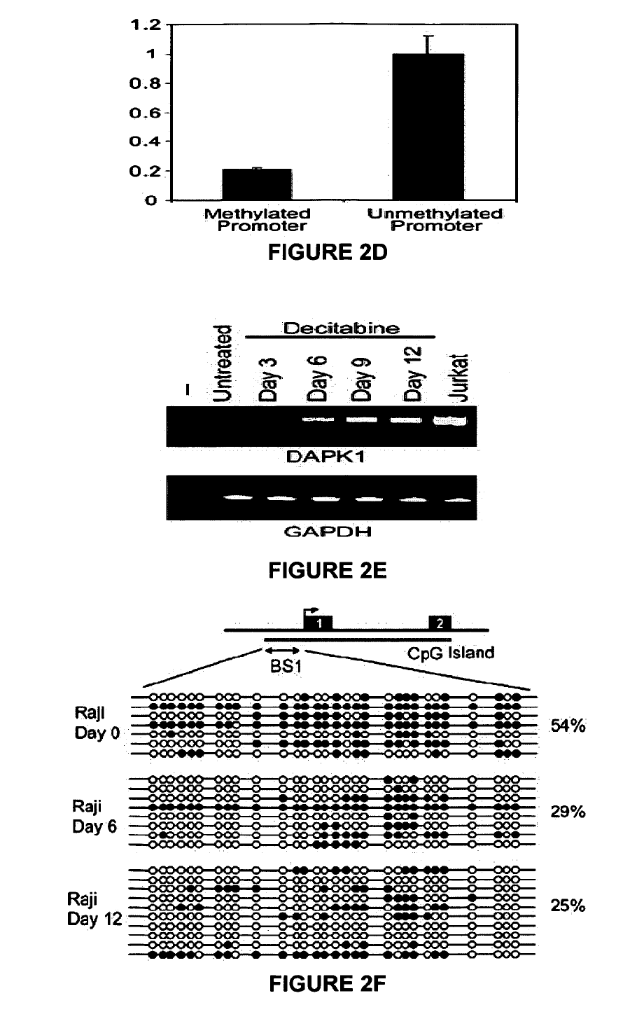

To study the relevance of promoter methylation for DAPK1 expression, we developed luciferase reporter constructs to test DAPK1 promoter activity in Jurkat cells. DAPK1 promoter construct #2 (c.1-1545 to c.1-1151 bp), covering bisulfite region A1 (displaying extensive promoter methylation; FIG. 1), showed a thirty-fold increase in the reporter activity relative to the vector control (FIG. 2A).

Semi-quantitative RT-PCR analysis of CD19+ CLL samples used in FIG. 1A, showed reduced DAPK1 expression in all seven samples (FIG. 2B). Furthermore, DAPK1 expression in 50 unselected CLL cells also showed statistically significant (p<0.01) reduction in all but three CLL samples as compared to normal CD19+ B-cells (FIG. 2C). FIG. 2D shows luciferase assays in 293T cells with either methylated or unmethylated DAPK1construct #2. Error bars indicate SD.

Re-Expression of DAPK1 by 5-aza-2′-deoxycytidine (Decitabine) Treatment:

Next we treated Raji cells with 0.51.LM Decitabine for 3, 6, 9, and 12 days. RT-PCR for DAPK1 indicated that Decitabine treatment resulted in gradual up-regulation of DAPK1 expression, while untreated Raji cells did not show any detectable expression (FIG. 2E).

Bisulfite sequencing of the BSI region showed that 116 of the tested 210 CpG dinucleotides (55%) were methylated in untreated Raji cells. Treatment with Decitabine resulted in significantly (p<0.001) reduced DNA methylation with 29% (70/240) on day 6 and 25% (68/270) on day 12 (FIG. 2F).

DAPK1 Regulates Apoptosis in Lymphoid Cells:

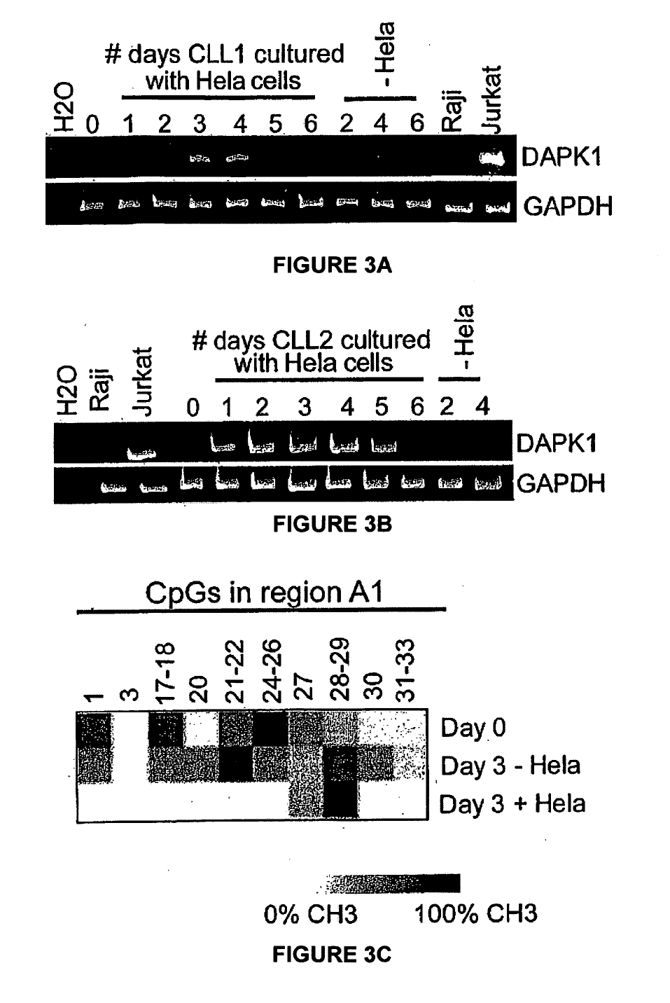

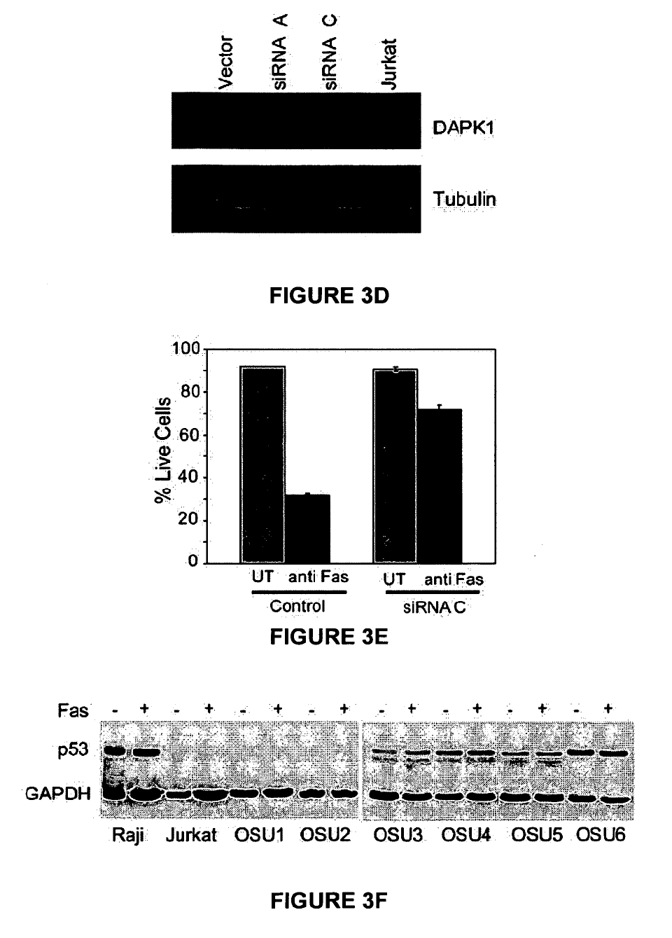

Frequent silencing of DAPK1 expression in CLL suggested a possible role in the etiology of CLL. To study the role of DAPK1 in primary CLL cells, we co-cultured primary CLL cells for 6 days with adherent HeLa cells in order to activate CD40 signaling and induce CD95 (Fas) (Dicker et al., 2005). RT-PCR analysis showed that exposure to CD40 resulted in induction of DAPK1 expression in two patient samples (CLL1 and CLL2; FIG. 3A and FIG. 3B) while the same was not observed in CLL cells cultured without HeLa. Both CLL1 and CLL2 showed increase in apoptosis when cultured with HeLa cells as reported by others (Dicker et al., 2005) (data not shown). DARK] promoter methylation was observed in the A1 region (FIG. 1A) in the CLL1 sample on days 0 and 3, while cells cultured with HeLa cells for 3 days showed reduced methylation at all CpG dinucleotides except CpGs 28-29 (FIG. 3C).

To further elucidate DAPK1 function in CD95-induced apoptosis, we stably transfected Jurkat cells with DAPK1 siRNA that resulted in its down-regulation (FIG. 3D). Cells transfected with vector alone or DAPK1 siRNA were treated with activating Fas and apoptosis was studied using annexin-V-FITC/Propidium Iodide (annexin/PI) flow cytometry.

Jurkat cells with inhibited DAPK1 expression showed a statistically significant increase in resistance to apoptosis compared to cells transfected with vector alone (p<0.001; FIG. 3E).

These data demonstrate that DAPK1 is involved in Fas-induced extrinsic apoptosis in lymphoid cells. Next, the inventors incubated 5×10⁷ cells from nine CLL patients with or without Fas-activating antibody (50 or 100 ng/ml) for 24 hr and examined apoptosis by annexin/PI flow cytometry. No significant effect on the percentage of nonapoptotic cells relative to the untreated control was seen. Immunoblot analysis to assess potential changes in pp53 expression following treatment showed no detectable differences (FIG. 3F). These experiments confirm that DAPK1 is a mediator of Fas-induced apoptotic signaling and loss of DAPK1 in CLL cells renders resistant to apoptosis.

These data demonstrate that DAPK1 is involved in Fas-induced extrinsic apoptosis in lymphoid cells.

A CLL Family with Linkage to Chromosome 9:

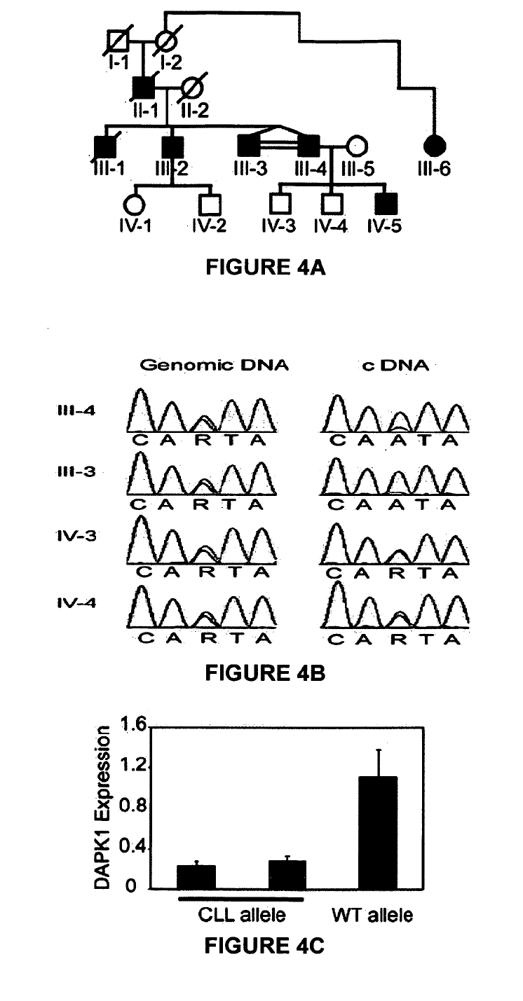

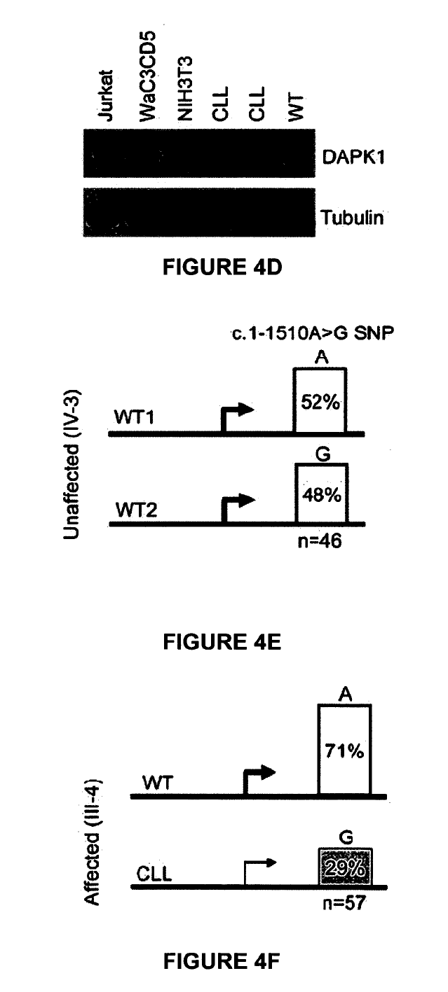

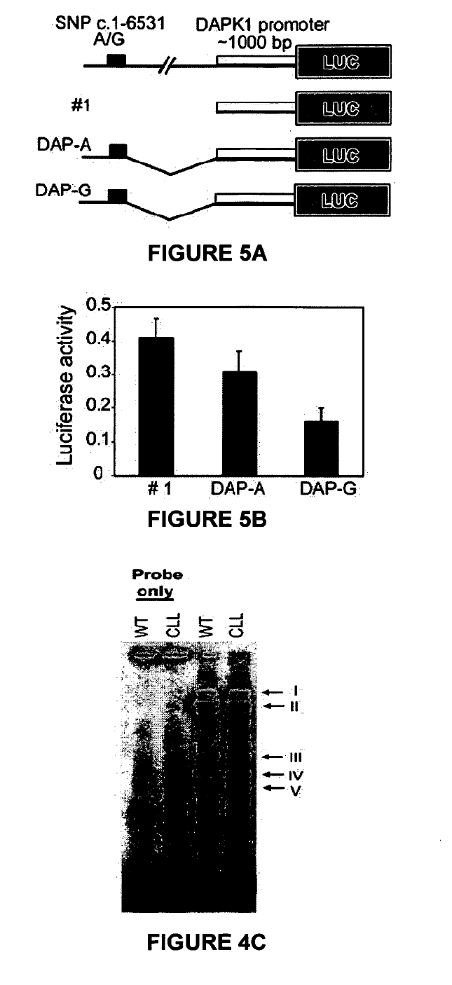

Lynch and colleagues previously documented an extended family in which a father and four sons were diagnosed with CLL (Lynch et al., 2002). We have since identified additional family members, both affected and unaffected (see FIG. 4A for extended pedigree). CLL was diagnosed in the father (II-1), his four sons (III-1, III-2, III-3, III-4), a grandson (IV-5) and a distant female relative (III-6). Genome-wide linkage analysis was performed using samples III-2, III-3, III-4, IV-3, IV-4 and IV-5 with a panel of 400 microsatellite markers. This identified a region on chromosome 9 between markers D9S175 to D9S1776 with the highest non-parametric linkage (NPL) score of 0.96. The next highest scores were less than 0.42. High-resolution genotyping identified a common haplotype of 707 kb in all affected family members for which samples were available. This locus in the presumed CLL haplotype includes 3 known genes (DAPK1, CTSL and CCRK) and 11 predicted genes

Based on the epigenetic data indicating frequent loss of DAPK1 expression, the inventors herein now believe that DAPK1 is a predisposing gene mutated in this family. To investigate, DAPK1 was sequenced from DNA of skin fibroblasts of four affected (III-2, 111-3, 111-4 and IV-5) as well as two unaffected family members (IV-3 and IV-4) and compared to the genomic sequence (NM_{—}004938). Although six DNA variants were detected (one in exon 3, two in exon 16, and three in exon 26), none was unique for the CLL haplotype. These variants were useful in subsequent RT-PCR and sequence analysis of DAPK1 and showed that gene expression was highly reduced in one allele (CLL allele) in CLL patient samples 111-4 and 111-3 whereas both alleles were expressed in unaffected individuals (FIG. 4B).

Allelic Expression Imbalance in DAPK1 in Affected Family Members:

As a next step we developed monochromosomal mouse-human hybrid clones containing either the WT (one clone) or the CLL (two clones) chromosome 9 from patient 111-4. Re-sequencing of DAPK1 exonic and mRNA sequences confirmed our earlier results, revealing no mutation in the transcribed region or the splice sites. Quantitative RT-PCR (FIG. 4C) and Western blotting (FIG. 4D) showed reduced DAPK1 expression from the two clones containing the CLL chromosome (25% or less) when compared to the clones containing the WT chromosome (100%). Reduced DAPK1 expression from the CLL allele was further confirmed in diploid cells. We cloned RT-PCR products containing exon 16 and comprising informative SNP c.1510G>A from an unaffected (IV-3) and an affected (III-4) family member.

Individual clones were genotyped by PCR using allele-specific primers or SSCP. As shown in FIG. 4E, in individual IV-3 the ratio of A to G clones was close to the expected ratio of 1:1, while in individual III-4, the number of A-clones (WT allele) was approximately 4 times that of the G clones (CLL allele, FIG. 4F) and this difference was statistically significant (p<0.01).

This was further confirmed by a quantitative single nucleotide primer extension (SnuPE) assay to quantify the mRNA levels from each allele. Genomic DNA and cDNA from affected and unaffected family members was amplified including SNP c.1608C>T. Next, fluorescently-labeled primers, specific for either one of the alleles, were used in a primer extension reaction and the intensities were quantified by comparing the ratios of the two alleles in cDNA and gDNA samples. The unaffected individual IV-4 showed a gDNA/cDNA ratio of 0.95 while in the affected individual III-4 the ratio was 0.36 as shown in FIG. 4F.

Detection of a Mutation by DAPK1 Genomic Sequencing:

Since the inventors herein now believe that the CLL allele specific repression might be due to a germline mutation in DAPK1 regulatory sequences, the inventors extended the genomic sequencing efforts (following the approach developed for the MHC haplotype project (Stewart et al., 2004)). BAC clones derived from both the affected and the unaffected allele of family member III-3 were generated and two overlapping BAC clones for the CLL (CR956620, CR956432) and the WT allele (CT009543, CR974482) were chosen for shotgun sequencing. Approximately 400 kbp were sequenced for each allele, extending from 45 kb upstream to 100 kb downstream of DAPK1, covering the entire gene. The sequence differences between the WT and CLL alleles were tabulated and compared with the published genomic sequence. No major rearrangements were detected in the sequence. In total 281 single nucleotide differences were observed between the two alleles, out of which 162 were reported SNPs and 87 were located within repeat elements. We further eliminated 28 of the remaining 32 SNPs as candidate mutations since they occurred in additional controls tested. One CLL specific SNP, c.1-6531A>G, was not found among 386 control samples from the US (n=281) and from Northern Europe (n=102). Screening of 263 CLL cases from the US (n=129) and from Northern Europe (n=134) identified one additional CLL sample from Scandinavia with SNP c.1-6531A>G.

HOXB7 Represses DAPK1 Transcription:

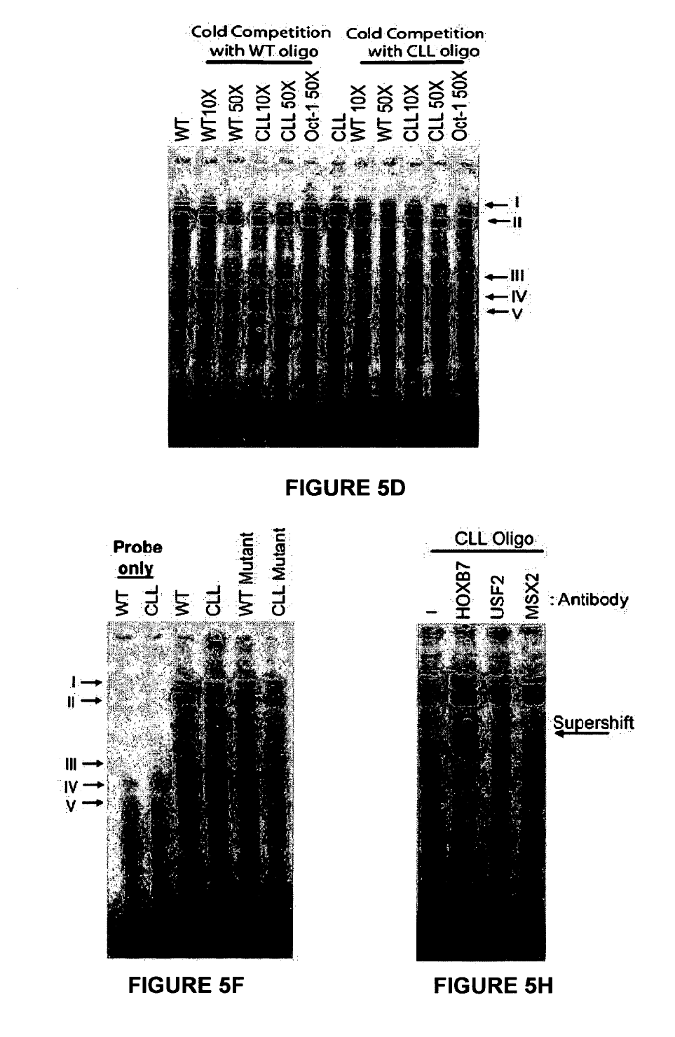

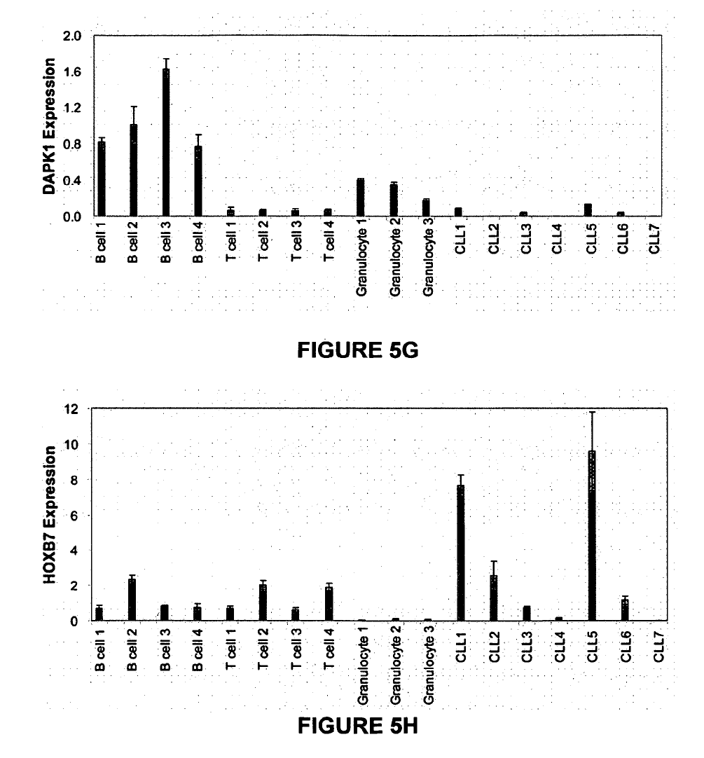

Since the CLL allele shows reduced expression, the inventors herein now believe that the mutation should facilitate suppressor sequences activity for the DAPK1 promoter. To study the effect of c.16531A>G on DAPK1 transcription, corresponding CLL and WT luciferase reporter constructs were designed (FIG. 5A). A 357 by fragment including either c.1-6531A (DAP-A) or c.16531G (DAP-G) was ligated upstream into luciferase construct #1 containing the DAPK1 promoter (see experimental procedures herein). Transcription from the DAP-A and DAP-B construct was suppressed by 39% and 70%, respectively, compared to control construct #1 (FIG. 5B). This suggested that both sequences contain a suppressor element; however the CLL derived sequences displayed stronger effects.

To identify the potential suppressor molecule that binds at c.1-6531A>G, an electrophoretic mobility shift assay (EMSA) was performed using oligonucleotides with either the WT oligo (A) or the CLL oligo (G). Using nuclear extracts from Jurkat cells, multiple bands were observed with both oligos (I to V; FIG. 5C).

Competition assays with unlabeled oligo confirmed specificity (FIG. 5D). The specificity of binding was further illustrated by mutating two base pairs on either side of A or G within the respective oligos.

FIG. 5E shows that while the mutation in the WT oligo resulted in elimination of band V, mutation of the CLL oligo resulted in elimination of bands IV and V. This result indicated that the WT and the CLL allele might bind similar complexes with different protein components. In addition, this result showed that protein binding at band IV is affected by the c.1-6531A>G mutation. Similar results were obtained using Raji nuclear extract (data not shown). Although EMSA is not a quantitative assay, the inventors observed in multiple independent experiments that the intensity of band IV with the CLL oligo was stronger than with the WT oligo hinting at a differential binding strength (FIGS. 5C, 5D and 5E).

Transcription factor binding site prediction suggested that HOX family proteins might have differential affinity to A or G basepair at the c.1-6531 SNP. To investigate, the inventors performed supershift assays using three HOX family proteins (HOXB7, MSX1 and MSX2). Only HOXB7 antibody induced a clear supershift, suggesting that HOXB7, or another closely related HOX protein, interacts with this site (FIG. 5F).

Next, the inventors tested DAPK1 and HOXB7 expression in B cells, T cells, and granulocytes of three health donors and selected B cells from CLL patients. DAPK1 expression was highest in normal B cells, and about one-fifth in granulocytes, respectively (FIG. 5G). Expression of DAPK1 was further reduced (on not detectable) in selected CLL cells. HOXB7 was expressed equally in B and T cells, however much reduced in granulocytes. HOBX7 was variably expressed in selected CD19+ CLL cells with several samples showing increased expression (FIG. 5H).

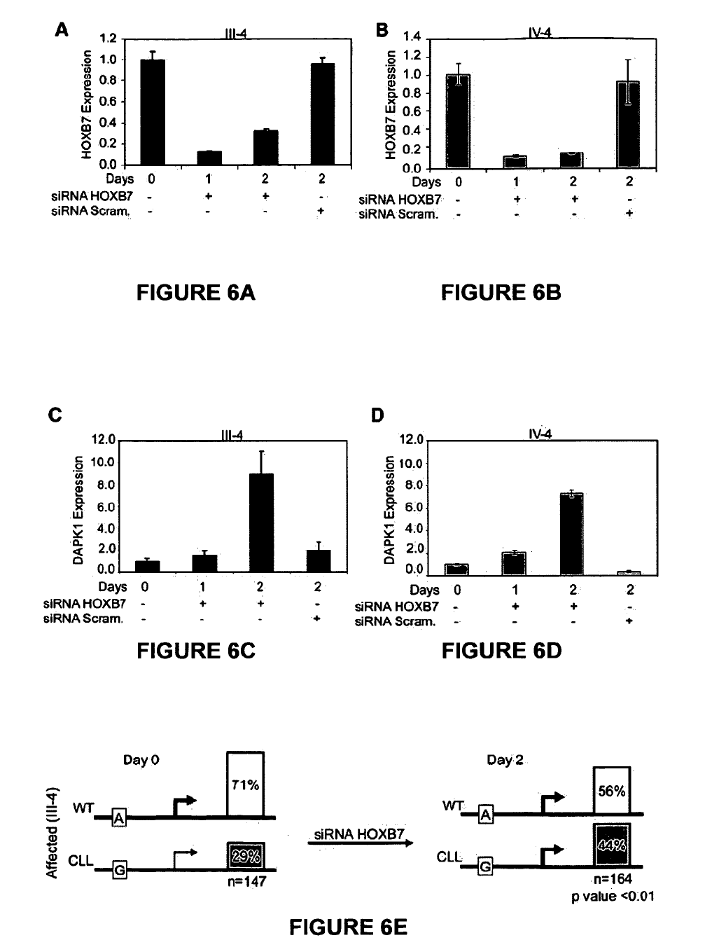

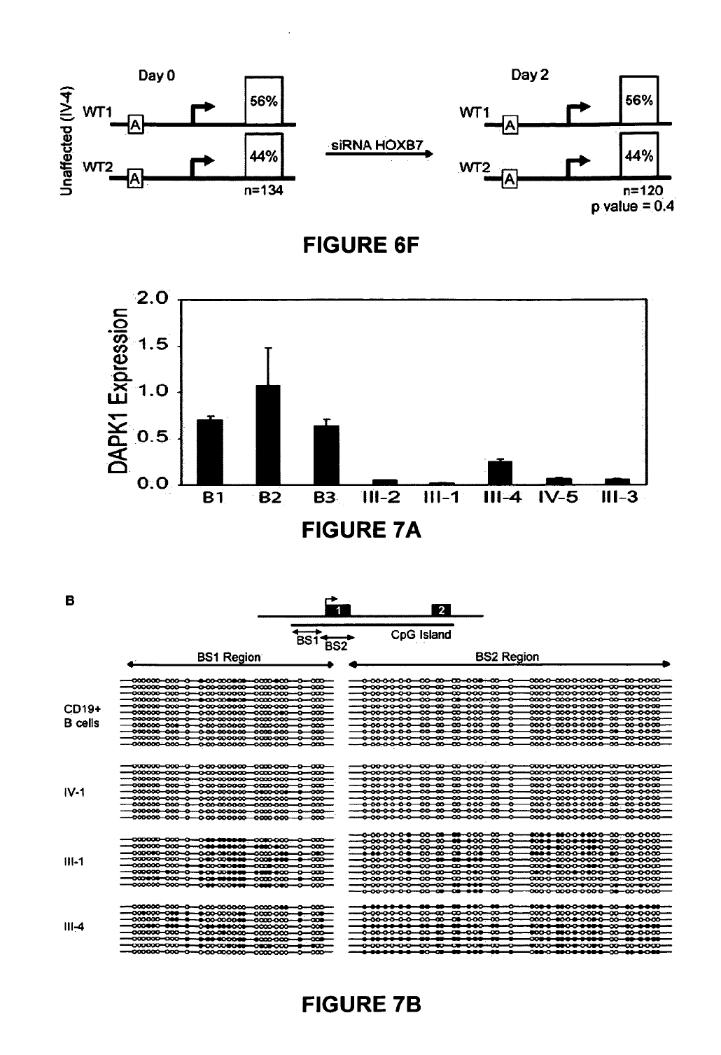

To explore if HOXB7 affects DAPK1 expression, we transfected skin fibroblasts from an affected (III-4) and an unaffected family member (IV-4) with HOXB7 siRNA. Quantitative RT-PCR showed HOXB7 down regulation in both samples within one day of transfection, while scrambled siRNA did not show this down regulation (FIGS. 6A and 6B).

Conversely, DAPK1 up regulation in HOXB7 siRNA transfected fibroblasts was observed in III-4 and IV-4, albeit at different levels. In an affected member, DAPK1 upregulation was 9-fold while in an unaffected member it was 5-fold compared to the untransfected fibroblast (FIGS. 6C and 6D). In addition, allele specific expression analysis by genotyping cloned RT-PCR products showed that activation of the CLL allele was significantly higher than activation of the WT allele supporting the notion that suppression by HOXB7 is higher in the CLL allele than in the WT allele. FIG. 6E.

DAPK1 Promoter Methylation in CLL Cells of Affected Family Members:

The results indicated that expression of the CLL allele was reduced to 25% of the WT allele in affected family members. Thus, total DAPK1 expression was reduced to approximately 60% of that of normal levels. The inventors now believe that additional DAPK1 repression during the progression of CLL followed due to promoter methylation in these neoplastic cells. Indeed, DAPK1 expression in peripheral blood cells of five affected family members was remarkably down-regulated when compared to three normal CD19+ cell controls (FIG. 7A). Bisulfite sequencing of peripheral blood DNA from affected (III-1 and III-4) and unaffected family members (IV-1) for both the BS I and the BS2 region showed that both regions were highly methylated in two affected family members (FIG. 7B). Altogether, these data show that DAPK1 expression in the affected family members is significantly reduced due to a combination of epigenetic and genetic aberrations.

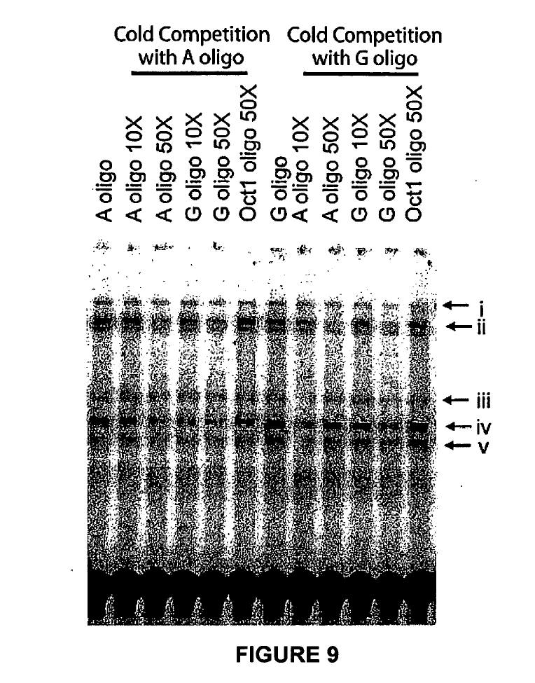

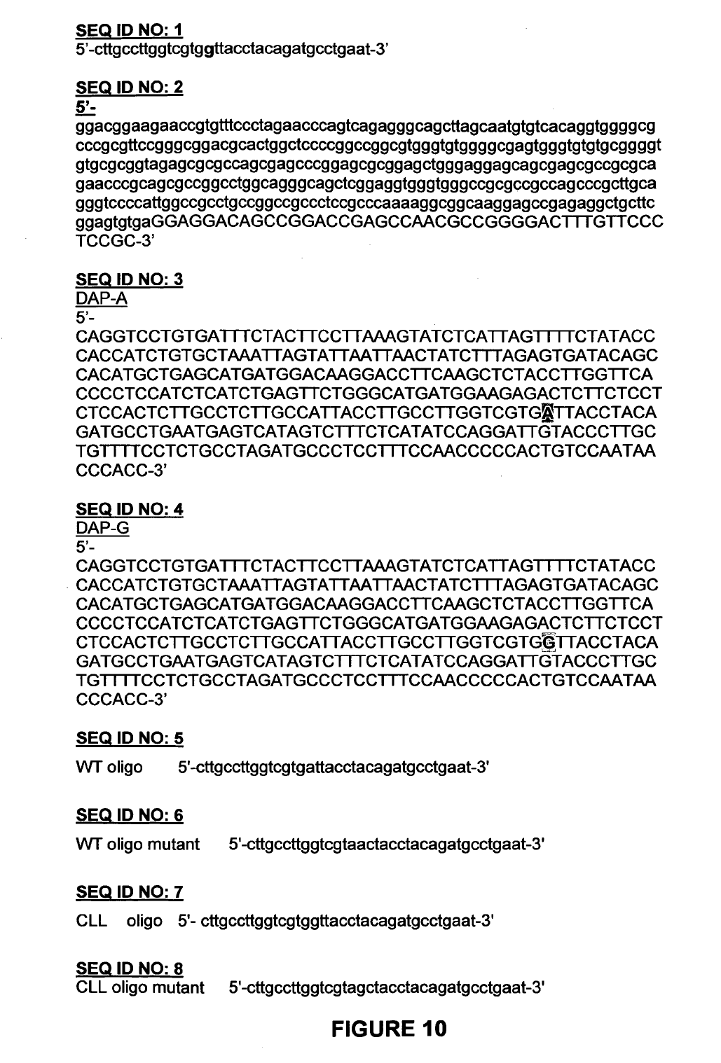

It is also to be noted that FIG. 9 shows cold competition with A oligo and cold competition with G oligo at 10× and 50×.

Discussion:

In a broad aspect, there is now shown herein the identification of DAPK1 as a tumor suppressor gene in CLL. While linkage studies did not provide sufficient resolution, for the first time the inventors were able to include epigenetic data to pinpoint a candidate gene in a CLL family. The inventors identified a mutation, predisposing to CLL, in the regulatory region of DAPK1 in a large family with seven affected individuals. This mutation enhances the binding affinity of transcription factor HOXB7, and results in down-regulation of DAPK1 transcription.

Importantly, DAPK1 is not only a target in familial CLL, but is also inactivated in the majority of sporadic cases of CLL by epigenetic mechanisms. The inventors' findings provide new insight into the extrinsic and intrinsic pathways of apoptosis, in which DAPK1 participates, and highlights the importance of normal DAPK1 expression in normal B-cells.

Furthermore, this invention demonstrates the need to integrate genetic and epigenetic data for the successful discovery of predisposing genes in cancer. Interestingly it has long been known that some high-penetrance genes (e.g. MLH1, BRCA1, p16, etc.) identified in familial cancers are also frequently silenced by epigenetic mechanisms in sporadic cancers (Baylin et al., 1998; Esteller, 2002). The role of DAPK1 in CLL appears to bear similarity to that of the mismatch repair gene MLH1 in colon cancer. Inactivating germline mutations of MLHI cause strong hereditary disposition to the Hereditary Nonpolyposis Colorectal Cancer syndrome. Of all colon cancers, approximately 1% are due to such mutations. However, additional 12-16% of sporadic colon cancers are due to silencing of MLHJ by acquired promoter hypermethylation (Lynch and de la Chapelle, 2003).

DAPK1 is an actin-filament associated, calcium calmodulin-dependent, serine/threonine kinase that promotes apoptosis in response to various stimuli including Fas, INF-y and TNF-a. (Bialik and Kimchi, 2006). Increased DAPK1 expression leads to death-associated cellular alterations and cell morphology changes (Cohen et al., 1997). DAPK1 silencing in other human malignancies is mediated by promoter methylation, associated with chromatin changes (Esteller, 2003; Gustafson et al., 2004; Hou et al., 2006; Neuhausen et al., 2006; Toyooka et al., 2003; Yegnasubramanian et al., 2004). With the exception of three homozygous deletions in soft tissue leiomyosarcoma, there are currently no reports of mutations in DAPK1 in any other tumor type (Kawaguchi et al., 2004). The DAPK1 promoter contains TGF-f3 response elements as well as p53 binding sites (Martoriati et al., 2005). DAPK1 overexpression results in upregulation of p53, suggesting a signaling feedback loop where DAPK1 and p53 regulate each other's expression. DAPK1 suppresses cMYC and E2F induced cell transformation by activating p19A1/p53 dependent apoptosis, and also blocks tumor metastasis in vivo (Inbal et al., 1997; Raveh et al., 2001). DAPK1 inhibits extracellular signal-regulated kinase (ERK) activity, and counteracts its survival signal (Chen et al., 2005), and promotes cytochrome c release from the mitochondria in response to TGF-13 induced apoptosis (Jang et al., 2002).

While the observed frequent silencing of DAPK1 by promoter methylation in CLL could be one of the events required by the leukemic cells to escape cell death mediated by either the intrinsic or extrinsic pathways of apoptosis (Bannerji and Byrd, 2000), until the present invention, there has not been any discovery that DAPK1 is involved in apoptotic signaling in B-cells.

CLL cells express CD40 on their surface and undergo activation upon interaction with CD40 ligand expressing cells, such as HeLa cells (Buhmann et al., 2002). CD40 activation induces expression of several immune accessory molecules, turns CLL cells into antigen presenting cells and also upregulates expression of CD95 (Fas), a member of the tumor necrosis factor (TNF) receptor family, which triggers apoptosis when engaged by the Fas ligand (Dicker et al., 2005). Indeed, CLL cells cultured with HeLa showed susceptibly to Fas induced apoptosis, upregulation of DAPK1 message, and simultaneous demethylation at its promoter suggesting a direct link between CLL cell death and DAPK1 expression. Modulating DAPK1 expression in Jurkat cells using siRNA provided an additional proof showing its direct involvement in apoptosis.

Past efforts have identified numerous affected genes in CLL; most of these studies relied on the identification of genes affected by genetic alterations, mainly deletions, which are frequently observed in sporadic CLLs (Byrd et al., 2006; Mehes, 2005; Stilgenbauer and Dohner, 2005). For example, deletion of 13q14 is commonly seen in up to 50% of sporadic CLL (Dohner et al., 2000). Several candidate tumor suppressor genes from this region have been proposed including miR15 and miR16, two microRNAs targeting the oncogene BCL2 (Bullrich et al., 2001; Calin et al., 2002; Cimmino et al., 2005; Hammarsund et al., 2004; Mertens et al., 2002). Other candidate genes include p53 (17p13) (Lin et al., 2003) and ATM (11g22) (Stankovic et al., 2002).

Mapping efforts in CLL families have been relatively unsuccessful in identifying genes predisposing to CLL. Several candidate genes and predisposing polymorphisms have been proposed including, ARLTSI, a gene that resides on chromosome 13q14 (Calin et al., 2005) and P2RX7, on chromosome 12q24 (Wiley et al., 2002). However, in follow-up studies this predisposition was controversial (Dao-Ung et al., 2004; Sellick et al., 2006; Thunberg et al., 2002).

The linkage results can be reconciled with the inventors' findings regarding the role of DAPK1. First, weak evidence of linkage to the DAPK1 region was seen by Goldin et al. (2003), however the region was negative in a follow-up study (Sellick et al., 2005). This suggests that heritable germline mutations of DAPK1 itself may not underlie the disease in many of the larger CLL families that have contributed to the previous linkage results. Second, the existence of multiple linkage peaks, none or few of which are statistically significant, suggests considerable locus heterogeneity or the existence of multiple mutated genes as recently proposed in an association study using nonsynonymous SNPs in candidate genes (Caporaso, 2006; Rudd et al., 2006). Third, this picture is compatible with the notion of multifactorial, or even multigenic causation. Fourth, since the inventors' results implicate down regulation of DAPK1 not only as the primary susceptibility factor in a large family, but also as a major event in sporadic CLL, it is quite possible that trans-acting factors, such as RNA genes (Calin and Croce, 2006; Esquela-Kerscher and Slack, 2006), play important roles in the deregulation of DAPK1, and other crucial genes, contributing to CLL. It was previously reported that such transacting genes show Mendelian inheritance (Morley et al., 2004), which suggests that they may account for a previously demonstrated linkage to multiple loci, however these putative genes need to be explored.

The occurrence of reduced DAPK1 expression in combination with frequent promoter methylation in the CLL cells in the family is intriguing. It is possible that reduced expression itself becomes a trigger for DAPK1 promoter methylation, which is initiated by transcriptional silencing (or reduced expression), followed by chromatin condensation and histone tail modifications, and finally by DNA methylation. This sequence of gene silencing events has been shown for target genes in the estrogen receptor signaling pathway in breast cancer cells (Leu et al., 2004) and for glutathione S-transferase in prostate cancer cells (Stirzaker et al., 2004).

To explain the frequent occurrence of promoter methylation in sporadic CLL, while not wishing to be bound by theory, the inventors herein now believe that DAPK1 silencing due to modulation of upstream signals. One of these signals, HOXB7, was identified herein. HOXB7 is a homeobox containing transcription factor mediating a variety of developmental processes, including hematopoietic differentiation and lymphoid development (Lill et al., 1995; Shen et al., 1989).

While not wishing to be bound by theory, the inventors herein also now believe that DAPK1 may at the end of the signaling cascade and modulation of any other upstream members could affect the downstream target, DAPK1. For example, activation of HOXB7 could, occur by activating mutations or loss of upstream repressors regulating this HOXB7. One of these factors, IkappaBalpha, an inhibitor of NF-kB activity, has been identified and it was shown that IkappaB-alpha enhances transcriptional activity of HOXB7 by direct interaction (Chariot et al., 1999). An alternative scenario that may explain frequent targeting of DAPK1 promoter by aberrant DNA methylation may be the aberrant recruitment of DNA methyltransferase activity to the DAPK1 promoter by oncogenic proteins (Brenner et al., 2005; Di Croce et al., 2002).

The inventors have identified DAPK1 as a novel tumor suppressor gene in CLL. The combination of linkage analysis and epigenetic studies demonstrate that down regulation of DAPK1 is common in CLL cells. The frequent silencing in sporadic CLL shows that aberrant silencing of DAPK1 is an early and required event in leukemogenesis. Screening of familial CLL cases for DAPK1 promoter methylation can help in early diagnosis of CLL, which is a late onset disease. In addition, as DNA methylation events are reversible, the inventors' discovery is useful for the development of novel treatment regimens in CLL involving epigenetic therapies for gene reactivation.

Experimental Procedures:

Patient Selection and Sample Collection:

Blood was obtained from patients with B-cell CLL through the CLL Research Consortium (CRC), from the Ohio State University and Uppsala University tissue banks. CLL patients from OSU and CRC consortium and healthy volunteers provided written informed consent using Institutional Review Board-approved protocols. Written consent for Uppsala samples was provided according to the declaration of Helsinki. Seven CD 19+ selected CLL samples were obtained by positive selection using magnetic beads conjugated to anti-CD19 (MACS, Miltenyi Biotec, Auburn Calif.). The resulting cells are at least 95% CD19 positive. All patients had immunophenotypically defined CLL as outlined by the modified 96 NCI criteria (Cheson et al., 1996) and for Uppsala samples, according to WHO classification. The CLL cell line WaC3CD5, described previously (Wendel-Hansen et al., 1994), Jurkat, Raji and lymphoblastoid cell lines were incubated (37° C. and 5% C02) in RPMI 1640 supplemented with 10% FBS (HyClone Laboratories, Logan, Utah), 2 mM L-glutamine, 100 U/ml penicillin, and 100 μg/ml streptomycin (all from Invitrogen, Carlsbad Calif.). The skin fibroblasts from family 4532 were cultured in DMEM medium, supplemented with 16% FCS and 100 U/ml penicillin, and 100 μg/ml streptomycin. Raji cells were treated with Decitabine (5-aza-2′-deoxycytidine) (Sigma-Aldrich, St. Louis Mo.) at 0.5 μM concentration for 3, 6, 9 and 12 days.

DNA and RNA Extraction: DNA and RNA were extracted from the patient samples and cell lines as described previously (Rush et al., 2004).

MassARRAY system for Quantitative DNA Methylation: Quantitative high-throughput DNA methylation analysis was done as described elsewhere (Ehrich et al., 2005). In brief, the DNA was bisulfite treated using EZ-96 DNA methylation kit (Zymo Research, Canada). Primers designed for PCR did not include CpGs, which allowed amplification of both methylated as well as unmethylated DNA. The primer sequences are available upon request. PCR of bisulfite treated DNA converted uracil to thymidine. The PCR products are then transcribed to RNA and cleaved base specifically. The C/T variation appears as G/A variation in the cleavage product. The G/A variations result in a mass difference of 16Da per CpG site, which is detected by the matrix-assisted laser desorption ionization time-of-flight mass spectrometry (MALTDI-TOF-MS).

Semi-Quantitative Reverse Transcriptase PCR (RT-PCR):

Semi-quantitative RT-PCR was performed as described previously (Liu et al., 2006). Briefly, 1.51.tg of total RNA was used for reverse transcription using SUPERSCRIPT™ First-Strand Synthesis kit (Invitrogen). SYBRgreen PCR was done in triplicates with IQ SYBR Green Supermix (Bio-Rad, Hercules, Calif.) in a BioRad icycler. The expression data was analyzed by comparative Ct method. Ct represents the cycle number at which the fluorescent signal first exceeds the threshold. The ACt value was obtained by subtracting the Ct value of the internal control (GAPDH or RPL4) from the Ct value of the target gene.

Expression Vectors and Luciferase Reporter Assay:

The pGL3 basic vector (Promega, Madison, Wis.) was modified by introducing NotI and EcoRV restriction sites (Yu et al., 2005). These sites were used to ligate the PCR amplified DAPK1 promoter constructs of different length. To study the effect of SNP c.1-6531 A>G by reporter assay, monochromosomal mouse-human hybrid cells were used to amplify 357 bp regions with either A or the G allele and the PCR products were ligated into pGL3-promoter vector at Xhol site and the SV40 promoter was replaced by DAPK1 promoter region (c.1-2215 to c.1-1151bp) at BglII and HindIII site to create DAP-G or DAP-A constructs. All constructs were confirmed by sequencing. For transfection into Jurkat cells a density of 1.5×105 cells/well were used in a 24-well plate with no serum for 2 hrs. After two hours 1 μg plasmid pGL3 vector and 20 ng of pRL-TK internal control vector (Promega) were co-transfected into cells using Polyethylenimine (Polysciences, Warrington, Pa.). After 4 hours, RPMI media with 10% FCS was added to each well to make up the volume to 1 ml. The cells were further incubated for 48 hours and the luciferase assay was performed according to manufactures instructions (Promega). Luciferase activity was normalized using pRL-TK activity. Each experiment was performed in triplicate.

Bisulfate Genomic Sequencing:

One microgram of genomic DNA was treated with sodium bisulfate according to published protocols (Herman et al., 1996). Regions BS1 and BS2 were amplified (primers are available upon request). The PCR product was purified using the Qiagen Gel Extraction kit (Qiagen, Chatsworth, Calif.) according to the manufacturer's protocol and subcloned using the TOPO TA-Cloning kit (Invitrogen) followed by sequencing of 8-10 clones for each sample using ABI big dye technology.

Chromatin Immunoprecipitation (ChIP) Assay:

Chromatin immunoprecipitation was carried out using the ChIP assay kit (Upstate Biotechnology, Lake Placid, N.Y.), as described previously (Tada et al., 2006). For immunoprecipitation, the antibodies used were rabbit polyclonal anti-acetylated H3-K9, anti-acetylated H3 and anti-acetylated H4 antibodies (Upstate Biotechnology, Lake Placid, N.Y.). Five micrograms of each antibody was used for immunoprecipitation and no antibody or 5 ug of rabbit IgG was used as negative controls. SYBR green semiquantitative PCR was performed as described before for the quantitation. Fold difference was calculated for each cell line relative to the negative control rabbit IgG.

siRNA Transfection:

For stable transfection of DAPK1 siRNA into Jurkat cells, pRS vector with different DAPK1 siRNA inserts (siRNA A and C) from OriGene, Rockville, Md. were used. Ten micrograms of pRS-DAPK1 siRNA A, siRNA C construct or pRS vector alone were transfected into the amphotropic Pheonix packaging cell line (60% confluent) using Superfect (Qiagen). Virus-containing medium was collected from the Pheonix cells after 48 hrs, and cell debris was removed by centrifugation. One milliliter of fresh medium plus 1 ml of infectious medium, containing either pRS-DAPK siRNA A, siRNA C or pRS vector alone were added to 2-5×105 Jurkat cells/well, and the 6 well plate was centrifuged at 2300 rpm for 90 min at RT in a Beckman centrifuge GPH, Rotor 3.7. Following the centrifugation step, 2 ml of fresh medium was added and 48 hrs after incubation in 5% C02, cells were selected for puromycin resistance using medium supplemented with 2 ug/ml Puromycin (Sigma, St Louis, Mo.).

For transcient transfection of HOXB7 siRNA (Ambion, Austin, Tex.) into fibroblast cells, 2×105 cells were plated and 24 hrs later cells were transfected using Polyethylenimine (Polysciences, Warrington, Pa.) as described before.

Apoptosis and Flow Cytometric Studies:

Apoptosis studies were done as described previously (Johnson et al., 2005). Briefly, Jurkat cells stably transfected with pRS vector control or pRS-DAPK siRNA C were treated with 100 ng/ml anti-activating Fas antibody (Upstate) for 16 hrs, and resuspended in binding buffer containing annexin V-fluorescein isothiocyanate (FITC) and propidium iodide according to the supplier's instructions (BD Biosciences, San Diego, Calif.), and assessed by flow cytometry using a Beckman-Coulter model EPICS XL cytometer (Beckman-Coulter, Miami, Fla.). Each sample was run in triplicate.

Immunoblot Analysis:

Immunoblotting was performed as described previously (Johnson et al., 2005). In brief, 100 μg of whole cell lysate was separated on an 8% PAGE gel and transferred to a nitrocellulose membrane (Hybond-ECL, Amersham Biosciences, Germany). Following the DAPK1 (Sigma-Aldrich, St. Louis Mo.) and a-tubulin antibody (Oncogene, Boston, Mass.) staining, the proteins were detected with chemiluminescent substrate (SuperSignal, Pierce, Rockford, Ill.).

Genomic Amplification and SSCP Analysis:

To screen the control and CLL samples for c.1-6531A>G mutation, genomic DNA was isolated (as described) and amplified using flanking primers. The amplified fragments were analyzed by Single Strand Conformation Polymorphism (SSCP) as previously described (Liechti-Gallati et al., 1999). Variant bands were re-amplified and used for direct sequencing on ABI Prism 3730 DNA analyzer (Applied Biosystems). For genotyping of the alleles for c.1-1510A>G SNP, the cDNA from unaffected III-4 and affected IV-4 samples with and without HOXB7 siRNA transfection were amplified and cloned.

SnuPE Assay for Allelic Variation in DAPK1 Expression:

The allele specific expression was studied as described previously (He et al., 2005). Genomic DNA and cDNA for the marker SNP was amplified using primers flanking the SNP c.1608C>T acid c.4037A>G. The two alleles were distinguished by fluorescently labeled nucleotide of the SNP in both genomic DNA and cDNA, and the peak area was used to determine the ratio of the two alleles. Primers sequences are available on request.

Electrophoretic Mobility Shift Assay (EMSA):

EMSA was performed as described elsewhere (Chen et al., 2002) with modifications. The oligonucleotides used were

WT oligo

[SEQ ID NO: 5]

5′-cttgccttggtcgtgattacctacagatgcctgaat-3′,
WT oligo mutant

[SEQ ID NO: 6]

5′-cttgccttggtcgtaactacctacagatgcctgaat-3′,
CLL oligo

[SEQ ID NO: 7]

5′-cttgccttggtcgtggttacctacagatgcctgaat-3′,
CLL oligo mutant

[SEQ ID NO: 8]

5′-cttgccttggtcgtagctacctacagatgcctgaat-3′.

The double-stranded oligonucleotides were endlabeled with [γ32P] ATP using T4 polynucleotide kinase enzyme (NEB). The free probe was removed by purification in G50 Sephadex spin columns. The binding reaction was conducted at room temperature for 20 min with 5 ug of nuclear extract, 40,000 dpm (0.08 to 0.4 ng) of radiolabelled oligonucleotide probe, in 5× Ficoll buffer (10 mM Tris (pH 7.5), 1 mM DTT, 1 mM EDTA and 4% Ficoll), 250 ng of poly(deoxyinosinic-deoxycytidylic acid) in 75 mM KCl and double distilled H20 to make the volume to 15u1. For supershift assay, 1 ug each of HOXB7, MSX2 (Cemines, Golden, Colo.) and USF2 (Santa Cruz Biotechnologies, Santa Cruz, Calif.) was added and the mixture was incubated at RT for additional 30 min. The DNA-protein complexes were fractionated by electrophoresis in 6% nondenaturating polyacrylamide gel, run in 0.5× Tris-borate-EDTA at 120 volts for 4 hrs at 4C. The gel was then dried on 3M Whatman paper and subjected to autoradiography. Radioactivity was visualized by autoradiography and was analyzed using STORM860 image analyzer (Amersham Biosciences, NJ). Nuclear extracts from Jurkat cells were made as described previously (Frissora et al., 2003).

This application relies on, and cites the disclosure of other patent applications and literature references. These documents are hereby incorporated by reference in their entireties for all purposes. The practice of the present invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, ligation, detection of hybridization using a label. Specific illustrations of suitable techniques can be had by reference to the examples herein. However, other equivalent conventional procedures can, of course, also be used. Such conventional techniques can be found in standard laboratory manuals such as Genome Analysis: A Laboratory Manual Series (Vols. I IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), all of which are herein incorporated in their entirety by reference for all purposes.

This application presents a detailed description of the preferred embodiments of the invention and their application. This description is by way of several exemplary illustrations, in increasing detail and specificity, and of the general methods of this invention. These examples are non-limiting, and related variants that will be apparent to one of skill in the art are intended to be encompassed by the appended claims. Also included are descriptions of embodiments of the data gathering steps that accompany the general methods.

Principles of the present invention are directed to the molecular analysis of CLL cells and to providing methods for obtaining information about consistent molecular alterations that advance both the understanding of the basic biology of such cells as well as the clinically relevant aspects of the molecular epidemiology of CLL. In one aspect, laser capture microdissection-derived RNA can be to be used on microarrays and that array hybridization coupled with hierarchical and non-hierarchical analysis methods provide powerful approaches for identifying candidate genes and molecular profiling associated with CCL.

Markers according to the present invention may include any nucleic acid sequence or molecule or corresponding polypeptide encoded by the nucleic acid sequence or molecule which demonstrates altered expression (i.e., higher or lower expression) in CLL samples relative to normal samples (i.e., non-cancerous samples).

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793 800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. Oligonucleotide and polynucleotide are included in this definition and relate to two or more nucleic acids in a polynucleotide.

Peptide: A polymer in which the monomers are alpha amino acids and which are joined together through amide bonds, alternatively referred to as a polypeptide and/or protein. In the context of this specification it should be appreciated that the amino acids may be, for example, the L-optical isomer or the D-optical isomer. Peptides are often two or more amino acid monomers long, and often 4 or more amino acids long, often 5 or more amino acids long, often 10 or more amino acids long, often 15 or more amino acids long, and often 20 or more amino acid monomers long, for example. Standard abbreviations for amino acids are used (e.g., P for proline). These abbreviations are included in Stryer, Biochemistry, Third Ed., 1988, which is incorporated herein by reference in its entirety for all purposes.

Array: An array comprises a solid support with peptide or nucleic acid probes attached to said support. Arrays typically comprise a plurality of different nucleic acid or peptide probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., Science, 251:767 777 (1991). Each of which is incorporated by reference in its entirety for all purposes. These arrays may generally be produced using mechanical synthesis methods or light directed synthesis methods which incorporate a combination of photolithographic methods and solid phase synthesis methods. Techniques for the synthesis of these arrays using mechanical synthesis methods are described in, e.g., U.S. Pat. Nos. 5,384,261, and 6,040,193 which are incorporated herein by reference in their entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated by reference in their entirety for all purposes. Arrays may be packaged in such a manner as to allow for diagnostics or other manipulation of in an all inclusive device, see for example, U.S. Pat. Nos. 5,856,174 and 5,922,591 incorporated in their entirety by reference for all purposes.

Gene expression monitoring is a useful way to distinguish between cells that express different phenotypes. For example, cells that are derived from different organs, have different ages, or different physiological states. In one embodiment, gene expression monitoring can distinguish between cancer cells and normal cells, or different types of cancer cells.

Expression profile: One measurement of cellular constituents that is particularly useful in the present invention is the expression profile. As used herein, an “expression profile” comprises measurement of the relative abundance of a plurality of cellular constituents. Such measurements may include RNA or protein abundances or activity levels. An expression profile involves providing a pool of target nucleic acid molecules or polypeptides, hybridizing the pool to an array of probes immobilized on predetermined regions of a surface, and quantifying the hybridized nucleic acid molecules or proteins. The expression profile can be a measurement, for example, of the transcriptional state or the translational state of the cell. See U.S. Pat. Nos. 6,040,138, 6,013,449 and 5,800,992, which are hereby incorporated by reference in their entireties for all purposes.

Transcriptional state: The transcriptional state of a sample includes the identities and relative abundances of the RNA species, especially mRNAs present in the sample. Preferably, a substantial fraction of all constituent RNA species in the sample are measured, but at least a sufficient fraction is measured to characterize the state of the sample. The transcriptional state is the currently preferred aspect of the biological state measured in this invention. It can be conveniently determined by measuring transcript abundances by any of several existing gene expression technologies.

Translational state: Translational state includes the identities and relative abundances of the constituent protein species in the sample. As is known to those of skill in the art, the transcriptional state and translational state are related.

The gene expression monitoring system, in a preferred embodiment, may comprise a nucleic acid probe array (such as those described herein), membrane blot (such as used in hybridization analysis such as Northern, Southern, dot, and the like), microwells, sample tubes, gels, beads or fibers (or any solid support comprising bound nucleic acids). See U.S. Pat. Nos. 5,770,722, 5,874,219, 5,744,305, 5,677,195 and 5,445,934, 5,800,992 which are expressly incorporated herein by reference in their entireties for all purposes.

The gene expression monitoring system may be used to facilitate a comparative analysis of expression in different cells or tissues, different subpopulations of the same cells or tissues, different physiological states of the same cells or tissue, different developmental stages of the same cells or tissue, or different cell populations of the same tissue.

Differentially expressed: The term differentially expressed as used herein means that a measurement of a cellular constituent varies in two samples. The cellular constituent can be either upregulated in the experiment relative to the reference or downregulated in the experiment relative to the reference. Differential gene expression can also be used to distinguish between cell types or nucleic acids. See U.S. Pat. No. 5,800,992.

One of skill in the art will appreciate that it is desirable to have nucleic acid samples containing target nucleic acid sequences that reflect the transcripts of interest. Therefore, suitable nucleic acid samples may contain transcripts of interest. Suitable nucleic acid samples, however, may contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

Transcripts, as used herein, may include, but are not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

In one embodiment, a sample is a homogenate of cells (e.g., blood cells), tissues or other biological samples. Preferably, such sample is a nucleic acid preparation, e.g., a total RNA preparation of a biological sample. More preferably in some embodiments, such a nucleic acid sample is the total mRNA isolated from a biological sample. Those of skill in the art will appreciate that the total mRNA prepared with most methods includes not only the mature mRNA, but also the RNA processing intermediates and nascent pre-mRNA transcripts. For example, total mRNA purified with a poly (T) column contains RNA molecules with poly (A) tails. Those poly A+ RNA molecules could be mature mRNA, RNA processing intermediates, nascent transcripts or degradation intermediates.

Biological samples may be of any biological tissue or fluid or cells. Frequently the sample will be a “clinical sample” which is a sample derived from a patient. Clinical samples provide rich sources of information regarding the various states of genetic network or gene expression. Some embodiments of the invention are employed to detect mutations and to identify the function of mutations. Such embodiments have extensive applications in clinical diagnostics and clinical studies. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

Other typical sources of biological samples are cell cultures where gene expression states can be manipulated to explore the relationship among genes. Various methods can be used to generate biological samples reflecting a wide variety of states of the genetic network.

In one embodiment, the level of expression of a marker for CLL is assessed by detecting the presence of a nucleic acid corresponding to the marker in the sample. In another embodiment, the level of expression of a marker for CLL is assessed by detecting the presence of a protein corresponding to the marker in the sample. In one preferred aspect, the presence of the protein is detected using a reagent which specifically binds to the protein, e.g., an antibody, an antibody derivative, and/or an antibody fragment.

Detection can involve contacting a sample with a compound or an agent capable of detecting a marker associated with oral cancer such that the presence of the marker is detected in the biological sample. One preferred agent for detecting marker RNA can be a labeled or labelable nucleic acid probe capable of hybridizing to marker RNA. The nucleic acid probe can be, for example, complementary to any of the nucleic acid markers of oral cancer disclosed herein, or a portion thereof, such as an oligonucleotide which specifically hybridizes marker RNA.

One useful agent for detecting a marker protein is a labeled or labelable antibody capable of binding to the marker protein. Antibodies can be polyclonal, or more preferably, monoclonal. An intact antibody, antibody derivative, or a fragment thereof (e.g., Fab or F(ab′).sub.2) can be used. The term “labeled or labelable”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Non-limiting examples of indirect labeling include detection of a primary antibody using a fluorescently labeled secondary antibody and end-labeling of a DNA probe with biotin such that it can be detected with fluorescently labeled streptavidin.

The detection methods described herein can be used to detect marker RNA or marker protein in a biological sample in vitro as well as in vivo. In vitro techniques for detection of marker RNA include, but are not limited to, Northern hybridizations and in situ hybridizations. In vitro techniques for detection of marker protein include, but are not limited to, enzyme linked immunosorbent assays (ELISAs), Western blots, immunoprecipitations, and immunofluorescence assays. Alternatively, marker protein can be detected in vivo in a subject by introducing into the subject a labeled antibody against the marker protein. For example, the antibody can be labeled with a radioactive marker whose presence and location in a subject can be detected by standard imaging techniques.

One of skill in the art would appreciate that it is desirable to inhibit or destroy RNase present in homogenates before homogenates can be used for hybridization. Methods of inhibiting or destroying nucleases are well known in the art. In some preferred embodiments, cells or tissues are homogenized in the presence of chaotropic agents to inhibit nuclease. In some other embodiments, RNases are inhibited or destroyed by heat treatment followed by proteinase treatment.

Methods of isolating total mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993).

In one embodiment, total RNA is isolated from a given sample using, for example, an acid guanidinium-phenol-chloroform extraction method followed by polyA+ mRNA isolation by oligo dT column chromatography or by using (dT)n magnetic beads (see, e.g., Sambrook et al., Molecular Cloning: A Laboratory Manual (2nd ed.), Vols. 1 3, Cold Spring Harbor Laboratory, (1989), or Current Protocols in Molecular Biology, F. Ausubel et al., ed. Greene Publishing and Wiley-Interscience, New York (1987) each hereby incorporated by reference in their entireties for all purposes). See also PCT/US99/25200 for complexity management and other sample preparation techniques, which is hereby incorporated by reference in its entirety for all purposes.

Frequently, it is desirable to amplify the nucleic acid sample prior to hybridization. One of skill in the art will appreciate that methods of amplifying nucleic acids are well known in the art and that whatever amplification method is used, if a quantitative result is desired, care must be taken to use a method that maintains or controls for the relative frequencies of the amplified nucleic acids to achieve quantitative amplification.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. A high density array may then be performed which includes probes specific to the internal standard for quantification of the amplified nucleic acid.

Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990)), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

Cell lysates or tissue homogenates often contain a number of regulators of polymerase activity. Therefore, the skilled practitioner typically incorporates preliminary steps to isolate total RNA or mRNA for subsequent use as an amplification template. One tube mRNA capture methods may be used to prepare poly(A)+ RNA samples suitable for immediate RT-PCR in the same tube (Boehringer Mannheim). The captured mRNA can be directly subjected to RT-PCR by adding a reverse transcription mix and, subsequently, a PCR mix.

In one particular embodiment, the sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, supra).

It will be appreciated by one of skill in the art that the direct transcription method described above provides an antisense RNA (aRNA) pool. Where aRNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning (see e.g., Palazzolo et al., Gene, 88: 25 36 (1990)).

Other analysis methods that can be used in the present invention include electrochemical denaturation of double stranded nucleic acids, U.S. Pat. Nos. 6,045,996 and 6,033,850, the use of multiple arrays (arrays of arrays), U.S. Pat. No. 5,874,219, the use of scanners to read the arrays, U.S. Pat. Nos. 5,631,734; 5,744,305; 5,981,956 and 6,025,601, methods for mixing fluids, U.S. Pat. No. 6,050,719, integrated device for reactions, U.S. Pat. No. 6,043,080, integrated nucleic acid diagnostic device, U.S. Pat. No. 5,922,591, and nucleic acid affinity columns, U.S. Pat. No. 6,013,440. All of the above patents are hereby incorporated by reference in their entireties for all purposes.

Also, laser dissection microscopy is one method that can be used in the present invention. Other techniques include L. Zhang et al., Science 276, 1268 (1997), Mahadevappa, M. & Warrington, J. A. Nat. Biotechnol. 17, 1134 1136 (1999) and Luo, L. et al. Nature Med. 5, 117 122 (1999) which are all hereby incorporated by reference in their entireties for all purposes.

As such, in another embodiment, the invention provides methods of assessing the efficacy of test compounds and compositions for treating CLL. The methods include identifying candidate or test compounds or agents (e.g., peptides, peptidomimetics, small molecules or other drugs) which have an inhibitory effect on CLL. Candidate or test compounds or agents which have an inhibitory effect on CLL are identified in assays that employ CLL cancer cells, such as an expression assay entailing direct or indirect measurement of the expression of a CLL marker (e.g., a nucleic acid marker or a protein marker). For example, modulators of expression of CLL markers can be identified in a method in which a cell is contacted with a candidate compound and the expression of CLL markers (e.g., nucleic acid markers and/or protein markers) in the cell is determined. The level of expression of CLL markers in the presence of the candidate compound is compared to the level of expression of CLL markers in the absence of the candidate compound. The candidate compound can then be identified as a modulator of expression of CLL based on this comparison.

In another aspect, the invention also encompasses kits for assessing whether a subject is afflicted with CLL, as well as kits for assessing the presence of CLL cells. The kit may comprise a labeled compound or agent capable of detecting CLL markers (e.g., nucleic acid markers and/or protein markers) in a biological sample, a means for determining the amount of CLL markers in the sample, and a means for comparing the amount of CLL markers in the sample with a standard. The compound or agent can be packaged in a suitable container. The kit can further comprise instructions for using the kit to detect CLL markers.

Those skilled in the art will recognize that in certain embodiments, the expression profiles from the reference samples will be input to a database. A relational database is preferred and can be used, but one of skill in the art will recognize that other databases could be used. A relational database is a set of tables containing data fitted into predefined categories. Each table, or relation, contains one or more data categories in columns. Each row contains a unique instance of data for the categories defined by the columns. For example, a typical database for the invention would include a table that describes a sample with columns for age, gender, reproductive status, expression profile and so forth. Another table would describe a disease: symptoms, level, sample identification, expression profile and so forth.

In another embodiment, the invention matches the experimental sample to a database of reference samples. The database is assembled with a plurality of different samples to be used as reference samples. An individual reference sample in one embodiment will be obtained from a patient during a visit to a medical professional. The sample could be, for example, a tissue, blood, urine, feces or saliva sample. Information about the physiological, disease and/or pharmacological status of the sample will also be obtained through any method available. This may include, but is not limited to, expression profile analysis, clinical analysis, medical history and/or patient interview. For example, the patient could be interviewed to determine age, sex, ethnic origin, symptoms or past diagnosis of disease, and the identity of any therapies the patient is currently undergoing. A plurality of these reference samples will be taken. A single individual may contribute a single reference sample or more than one sample over time. One skilled in the art will recognize that confidence levels in predictions based on comparison to a database increase as the number of reference samples in the database increases. One skilled in the art will also recognize that some of the indicators of status will be determined by less precise means, for example information obtained from a patient interview is limited by the subjective interpretation of the patient. Additionally, a patient may lie about age or lack sufficient information to provide accurate information about ethnic or other information. Descriptions of the severity of disease symptoms is a particularly subjective and unreliable indicator of disease status.

The database is organized into groups of reference samples. Each reference sample contains information about physiological, pharmacological and/or disease status. In one aspect, the database is a relational database with data organized in three data tables, one where the samples are grouped primarily by physiological status, one where the samples are grouped primarily by disease status, and one where the samples are grouped primarily by pharmacological status. Within each table the samples can be further grouped according to the two remaining categories. For example, the physiological status table could be further categorized according to disease and pharmacological status.

As will be appreciated by one of skill in the art, the present invention may be embodied as a method, data processing system or program products. Accordingly, the present invention may take the form of data analysis systems, methods, analysis software, and the like. Software written according to the present invention is to be stored in some form of computer readable medium, such as memory, hard-drive, DVD ROM or CD ROM, or transmitted over a network, and executed by a processor. The present invention also provides a computer system for analyzing physiological states, levels of disease states and/or therapeutic efficacy. The computer system comprises a processor, and memory coupled to said processor which encodes one or more programs. The programs encoded in memory cause the processor to perform the steps of the above methods wherein the expression profiles and information about physiological, pharmacological and disease states are received by the computer system as input. U.S. Pat. No. 5,733,729 illustrates an example of a computer system that may be used to execute the software of an embodiment of the invention. This patent shows a computer system that includes a display, screen, cabinet, keyboard, and mouse. The mouse may have one or more buttons for interacting with a graphic user interface. The cabinet preferably houses a CD-ROM or DVD-ROM drive, system memory and a hard drive which may be utilized to store and retrieve software programs incorporating computer code that implements the invention, data for use with the invention and the like. Although a CD is shown as an exemplary computer readable medium, other computer readable storage media including a floppy disk, a tape, a flash memory, a system memory, and a hard drive may be utilized.

Additionally, a data signal embodied in a carrier wave (e.g., in a network including the internet) may be the computer readable storage medium. The patent also shows a system block diagram of a computer system used to execute the software of an embodiment of the invention. The computer system includes a monitor, a keyboard, and a mouse. The computer system further includes subsystems such as a central processor, a system memory, a fixed storage (e.g., a hard drive), a removable storage (e.g., CD-ROM), a display adapter, a sound card, speakers, and a network interface. Other computer systems suitable for use with the invention may include additional or fewer subsystems. For example, another computer system may include more than one processor or a cache memory. Computer systems suitable for use with the invention may also be embedded in a measurement instrument. The embedded systems may control the operation of, for example, a GENECHIP®. Probe array scanner (also called a GENEARRAY® seamier sold by Agilent corporation, Palo Alto, Calif.) as well as executing computer codes of the invention.

Computer methods can be used to measure the variables and to match samples to eliminate gene expression differences that are a result of differences that are not of interest. For example, a plurality of values can be input into computer code for one or more physiological, pharmacological and/or disease states. The computer code can thereafter measure the differences or similarities between the values to eliminate changes not attributable to a value of interest.

Computer software to analyze data generated by microarrays is commercially available from Affymetrix Inc. (Santa Clara) as well as other companies. Affymetrix Inc. distributes GENECHIP®, now known as MicroArray suite, LIMS, Microdb, Jaguar, DMT, and other software. Other databases can be constructed using the standard database tools available from Microsoft (e.g., Excel and Access).

High-density oligonucleotide arrays are particularly useful for monitoring the gene expression pattern of a sample. In one approach, total mRNA isolated from the sample is converted to labeled cRNA and then hybridized to an array such as a GENECHIP® oligonucleotide array. Each sample is hybridized to a separate array. Relative transcript levels are calculated by reference to appropriate controls present on the array and in the sample.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

### REFERENCES

Any publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.
Amundadottir, L. T., Thorvaldsson, S., Gudbjartsson, D. F., Sulem, P., Kristjansson, K., Amason, S., Gulcher, J. R., Bjomsson, J., Kong, A., Thorsteinsdottir, U., and Stefansson, K. (2004). Cancer as a complex phenotype: pattern of cancer distribution within and beyond the nuclear family. PLoS Med 1, e65.
Bannerji, R., and Byrd, J. C. (2000). Update on the biology of chronic lymphocytic leukemia. Curr Opin Oncol 12, 22-29.
Baylin, S. B., Herman, J. G., Graff, J. R., Vertino, P. M., and Issa, J. P. (1998). Alterations in DNA methylation: a fundamental aspect of neoplasia. Adv Cancer Res 72, 141-196.
Bialik, S., and Kimchi, A. (2006). The Death-Associated Protein Kinases: Structure, Function, and Beyond. Annu Rev Biochem.
Brenner, C., Deplus, R., Didelot, C., Loriot, A., Vire, E., De Smet, C., Gutierrez, A., Danovi, D., Bernard, D., Boon, T., at al. (2005). Myc represses transcription through recruitment of DNA methyltransferase corepressor. Embo J 24, 336-346.
Buhmann, R., Kurzeder, C., Rehklau, J., Westhaus, D., Bursch, S., Hiddemann, W., Haferlach, T., Hallek, M., and Schoch, C. (2002). CD40L stimulation enhances the ability of conventional metaphase cytogenetics to detect chromosome aberrations in B-cell chronic lymphocytic leukaemia cells. Br J Haematol 118, 968-975.
Bullrich, F., Fujii, H., Calin, G., Mabuchi, H., Negrini, M., Pekarsky, Y., Rassenti, L., Alder, H., Reed, J. C., Keating, M. J., et al. (2001). Characterization of the 13q14 tumor suppressor locus in CLL: identification of ALT1, an alternative splice variant of the LEU2 gene. Cancer Res 61, 6640-6648.
Byrd, J. C., Gribben, J. G., Peterson, B. L., Greyer, M. R., Lozanski, G., Lucas, D. M., Lampson, B., Larson, R. A., Caligiuri, M. A., and Heerema, N. A. (2006). Select high-risk genetic features predict earlier progression following chemoimmunotherapy with fludarabine and rituximab in chronic lymphocytic leukemia: justification for risk-adapted therapy. J Clin Oncol 24, 437-443.
Calin, G. A., and Croce, C. M. (2006). Genomics of chronic lymphocytic leukemia microRNAs as new players with clinical significance. Semin Oncol 33, 167-173. Calin, G. A., Dumitru, C. D., Shimizu, M., Bichi, R., Zupo, S., Noch, E., Aldler, H., Rattan, S., Keating, M., Rai, K., et al. (2002). Frequent deletions and down-regulation of micro-RNA genes miR15 and miR16 at 13q14 in chronic lymphocytic leukemia. Proc Natl Acad Sci USA 99, 15524-15529.
Calin, G. A., Trapasso, F., Shimizu, M., Dumitru, C. D., Yendamuri, S., Godwin, A. K., Ferracin, M., Bernardi, G., Chatterjee, D., Baldassarre, G., et al. (2005). Familial cancer associated with a polymorphism in ARLTS1. N Engl J Med 352, 1667-1676.
Caporaso, N. (2006). Chips, candidate genes, and CLL. Blood 108, 415-416.
Chariot, A., Princen, F., Gielen, J., Melville, M. P., Franzoso, G., Brown, K., Siebenlist, U., and Bours, V. (1999). IkappaB-alpha enhances transactivation by the HOXB7 homeodomain-containing protein. J Biol Chem 274, 5318-5325.
Chen, C. H., Wang, W. J., Kuo, J. C., Tsai, H. C., Lin, J. R., Chang, Z. F., and Chen, R. H. (2005). Bidirectional signals transduced by DAPK-ERK interaction promote the apoptotic effect of DAPK. Embo J 24, 294-304.
Chen, L., Widhopf, G., Huynh, L., Rassenti, L., Rai, K. R., Weiss, A., and Kipps, T. J. (2002). Expression of ZAP-70 is associated with increased B-cell receptor signaling in chronic lymphocytic leukemia. Blood 100, 4609-4614.
Cheson, B. D., Bennett, J. M., Greyer, M., Kay, N., Keating, M. J., O'Brien, S., and Rai, K. R. (1996). National Cancer Institute-sponsored Working Group guidelines for chronic lymphocytic leukemia: revised guidelines for diagnosis and treatment. Blood 87, 4990-4997.
Cimmino, A., Calin, G. A., Fabbri, M., Iorio, M. V., Ferracin, M., Shimizu, M., Wojcik, S. E., Aqeilan, R. I., Zupo, S., Dono, M., at al. (2005). miR-15 and miR-16 induce apoptosis by targeting BCL2. Proc Natl Acad Sci USA 102, 13944-13949.
Cohen, O., Feinstein, E., and Kimchi, A. (1997). DAP-kinase is a Ca2+/calmodulin-dependent, cytoskeletal-associated protein kinase, with cell death-inducing functions that depend on its catalytic activity. Embo J 16, 998-1008.
Corcoran, M., Parker, A., Orchard, J., Davis, Z., Wirtz, M., Schmitz, O. J., and Oscier, D. (2005). ZAP-70 methylation status is associated with ZAP-70 expression status in chronic lymphocytic leukemia. Haematologica 90, 1078-1088.
Dao-Ung, L. P., Fuller, S. J., Sluyter, R., SkarRatt, K. K., Thunberg, U., Tobin, G., Byth, K., Ban, M., Rosenquist, R., Stewart, G. J., and Wiley, J. S. (2004). Association of the 1513C polymorphism in the P2X7 gene with familial forms of chronic lymphocytic leukaemia. Br J Haematol 125, 815-817.
Deiss, L. P., Feinstein, E., Berissi, H., Cohen, O., and Kimchi, A. (1995). Identification of a novel serine/threonine kinase and a novel 15-1cD protein as potential mediators of the gamma interferon-induced cell death. Genes Dev 9, 15-30.
Di Croce, L., Raker, V. A., Corsaro, M., Fazi, F., Fanelli, M., Faretta, M., Fuks, F., Lo Coco, F., Kouzarides, T., Nervi, C., et al. (2002). Methyltransferase recruitment and DNA hypermethylation of target promoters by an oncogenic transcription factor. Science 295, 1079-1082.
Dicker, F., Kater, A. P., Fukuda, T., and Kipps, T. J. (2005). Fas-ligand (CD 178) and TRAIL synergistically induce apoptosis of CD40-activated chronic lymphocytic leukemia B cells. Blood 105, 3193-3198.
Dohner, H., Stilgenbauer, S., Benner, A., Leupolt, E., Krober, A., Bullinger, L., Dohner, K., Bentz, M., and Lichter, P. (2000). Genomic aberrations and survival in chronic lymphocytic leukemia. N Engl J Med 343, 1910-1916.
Ehrich, M., Nelson, M. R., Stanssens, P., Zabeau, M., Liloglou, T., Xinarianos, G., Cantor, C. R., Field, J. K., and van den Boom, D. (2005). Quantitative high-throughput analysis of DNA methylation patterns by base-specific cleavage and mass spectrometry. Proc Natl Acad Sci USA 102, 15785-15790.
Esquela-Kerscher, A., and Slack, F. J. (2006). Oncomirs—microRNAs with a role in cancer. Nat Rev Cancer 6, 259-269.
Esteller, M. (2002). CpG island hypermethylation and tumor suppressor genes: a booming present, a brighter future. Oncogene 21, 5427-5440.
Esteller, M. (2003). Profiling aberrant DNA methylation in hematologic neoplasms: a view from the tip of the iceberg. Clin Immunol 109, 80-88.
Frissora, F., Chen, H. C., Durbin, J., Bondada, S., and Muthusamy, N. (2003). IFN-gamma-mediated inhibition of antigen receptor-induced B cell proliferation and CREB-1 binding activity requires STAT-1 transcription factor. Eur J Immunol 33, 907-912.
Goldgar, D. E., Easton, D. F., Cannon-Albright, L. A., and Skolnick, M. H. (1994). Systematic population-based assessment of cancer risk in first-degree relatives of cancer probands. J Natl Cancer Inst 86, 1600-1608.
Goldin, L. R., Ishibe, N., Sgambati, M., Marti, G. E., Fontaine, L., Lee, M. P., Kelley, J. M., Scherpbier, T., Buetow, K. H., and Caporaso, N. E. (2003). A genome scan of 18 families with chronic lymphocytic leukaemia. Br J Haematol 121, 866-873.
Goldin, L. R., Pfeiffer, R. M., Li, X., and Hemminki, K. (2004). Familial risk of lymphoproliferative tumors in families of patients with chronic lymphocytic leukemia: results from the Swedish Family-Cancer Database. Blood 104, 1850-1854.
Gustafson, K. S., Furth, E. E., Heitjan, D. F., Fansler, Z. B., and Clark, D. P. (2004). DNA methylation profiling of cervical squamous intraepithelial lesions using liquid-based cytology specimens: an approach that utilizes receiver-operating characteristic analysis. Cancer 102, 259-268.
Hammarsund, M., Corcoran, M. M., Wilson, W., Zhu, C., Einhorn, S., Sangfelt, O., and Grander, D. (2004). Characterization of a novel B-CLL candidate gene—DLEU7—located in the 13q14 tumor suppressor locus. FEES Lett 556, 75-80.
He, H., Olesnanik, K., Nagy, R., Liyanarachchi, S., Prasad, M. L., Stratakis, C. A., Kloos, R. T., and de la Chapelle, A. (2005). Allelic variation in gene expression in thyroid tissue. Thyroid 15, 660-667.
Herman, J. G., Jen, J., Merlo, A., and Baylin, S. B. (1996). Hypermethylation-associated inactivation indicates a tumor suppressor role for p151NK4B. Cancer Res 56, 722-727.
Hou, P., Ji, M., Yang, B., Chen, Z., Qiu, J., Shi, X., and Lu, Z. (2006). Quantitative analysis of promoter hypermethylation in multiple genes in osteosarcoma. Cancer 106, 1602-1609.
Inbal, B., Cohen, O., Polak-Charcon, S., Kopolovic, J., Vadai, E., Eisenbach, L., and Kimchi, A. (1997). DAP kinase links the control of apoptosis to metastasis. Nature 390, 180-184.
Jang, C. W., Chen, C. H., Chen, C. C., Chen, J. Y., Su, Y. H., and Chen, R. H. (2002). TGF-beta induces apoptosis through Smad-mediated expression of DAP-kinase. Nat Cell Biol 4, 51-58. Johnson, A. J., Smith, L. L., Zhu, J., Heerema, N. A., Jefferson, S., Mone, A., Greyer, M., Chen,
C. S., and Byrd, J. C. (2005). A novel celecoxib derivative, OSU03012, induces cytotoxicity in primary CLL cells and transformed B-cell lymphoma cell line via a caspase- and Bcl-2-independent mechanism. Blood 105, 2504-2509.
Kasper, D. L., and Harrison, T. R. (2005). Harrison's principles of internal medicine, 16th edn (New York: McGraw-Hill, Medical Pub. Division).
Katzenellenbogen, R. A., Baylin, S. B., and Herman, J. G. (1999). Hypermethylation of the DAP-kinase CpG island is a common alteration in B-cell malignancies. Blood 93, 4347-4353.
Kawaguchi, K., Oda, Y., Saito, T., Yamamoto, H., Takahira, T., Tamiya, S., Iwamoto, Y., and Tsuneyoshi, M. (2004). Death-associated protein kinase (DAP kinase) alteration in soft tissue leiomyosarcoma: Promoter methylation or homozygous deletion is associated with a loss of DAP kinase expression. Hum Pathol 35, 1266-1271.
Leu, Y. W., Yan, P. S., Fan, M., Jin, V. X., Liu, J. C., Curran, E. M., Welshons, W. V., Wei, S. H., Davuluri, R. V., Plass, C., et al. (2004). Loss of estrogen receptor signaling triggers epigenetic silencing of downstream targets in breast cancer. Cancer Res 64, 8184-8192.
Liechti-Gallati, S., Schneider, V., Neeser, D., and Kraemer, R. (1999). Two buffer PAGE system-based SSCP/HD analysis: a general protocol for rapid and sensitive mutation screening in cystic fibrosis and any other human genetic disease. Eur J Hum Genet 7, 590598.
Lill, M. C., Fuller, J. F., Herzig, R., Crooks, G. M., and Gasson, J. C. (1995). The role of the homeobox gene, HOX B7, in human myelomonocytic differentiation. Blood 85, 692-697.
Lin, K., Manocha, S., Harris, R. J., Matrai, Z., Sherrington, P. D., and Pettitt, A. R. (2003). High frequency of p53 dysfunction and low level of VH mutation in chronic lymphocytic leukemia patients using the VH3-21 gene segment. Blood 102, 1145-1146.
Liu, T. H., Raval, A., Chen, S. S., Matkovic, J. J., Byrd, J. C., and Plass, C. (2006). CpG island methylation and expression of the secreted frizzled-related protein gene family in chronic lymphocytic leukemia. Cancer Res 66, 653-658.
Lynch, H. T., and de la Chapelle, A. (2003). Hereditary colorectal cancer. N Engl J Med 348, 919-932.
Lynch, H. T., Weisenburger, D. D., Quinn-Laquer, B., Watson, P., Lynch, J. F., and Sanger, W. G. (2002). Hereditary chronic lymphocytic leukemia: an extended family study and literature review. Am J Med Genet 115, 113-117.
Martoriati, A., Doumont, G., Alcalay, M., Bellefroid, E., Pelicci, P. G., and Marine, J. C. (2005). dapkl, encoding an activator of a p19ARF-p53-mediated apoptotic checkpoint, is a transcription target of p53. Oncogene 24, 1461-1466.
Mehes, G. (2005). Chromosome abnormalities with prognostic impact in B-cell chronic lymphocytic leukemia. Pathol Oncol Res 11, 205-210.
Mertens, D., Wolf, S., Schroeter, P., Schaffner, C., Dohner, H., Stilgenbauer, S., and Lichter, P. (2002). Down-regulation of candidate tumor suppressor genes within chromosome band 13q14.3 is independent of the DNA methylation pattern in B-cell chronic lymphocytic leukemia. Blood 99, 4116-4121.
Morley, M., Molony, C. M., Weber, T. M., Devlin, J. L., Ewens, K. G., Spielman, R. S., and Cheung, V. G. (2004). Genetic analysis of genome-wide variation in human gene expression. Nature 430, 743-747.
Neuhausen, A., Florl, A. R., Grimm, M. O., and Schulz, W. A. (2006). DNA Methylation Alterations in Urothelial Carcinoma. Cancer Biol Ther 5.
Raval, A., Lucas, D. M., Matkovic, J. J., Bennett, K. L., Liyanarachchi, S., Young, D. C., Rassenti, L., Kipps, T. J., Greyer, M. R., Byrd, J. C., and Plass, C. (2005). TWIST2 Demonstrates Differential Methylation in Immunoglobulin Variable Heavy Chain Mutated and Unmutated Chronic Lymphocytic Leukemia. J Clin Oncol.
Raveh, T., Droguett, G., Horwitz, M. S., DePinho, R. A., and Kimchi, A. (2001). DAP kinase activates a p19ARF/p53-mediated apoptotic checkpoint to suppress oncogenic transformation. Nat Cell Biol 3, 1-7.
Risch, N. (2001). The genetic epidemiology of cancer: interpreting family and twin studies and their implications for molecular genetic approaches. Cancer Epidemiol Biomarkers Prey 10, 733-741.
Rosenwald, A., Wright, G., Leroy, K., Yu, X., Gaulard, P., Gascoyne, R. D., Chan, W. C., Zhao, T., Haioun, C., Greiner, T. C., et al. (2003). Molecular diagnosis of primary mediastinal B cell lymphoma identifies a clinically favorable subgroup of diffuse large B cell lymphoma related to Hodgkin lymphoma. J Exp Med 198, 851-862.
Rudd, M. F., Sellick, G. S., Webb, E. L., Catovsky, D., and Houlston, R. S. (2006). Variants in the ATM-BRCA2-CHEK2 axis predispose to chronic lymphocytic leukemia. Blood 108, 638644.
Rush, L. J., Raval, A., Funchain, P., Johnson, A. J., Smith, L., Lucas, D. M., Bembea, M., Liu, T.
Heerema, N. A., Rassenti, L., et al. (2004). Epigenetic profiling in chronic lymphocytic leukemia reveals novel methylation targets. Cancer Res 64, 2424-2433.
Sellick, G. S., Catovsky, D., and Houlston, R. S. (2006). Familial chronic lymphocytic leukemia. Semin Oncol 33, 195-201.
Sellick, G. S., Webb, E. L., Allinson, R., Matutes, E., Dyer, M. J., Jonsson, V., Langerak, A. W., Mauro, F. R., Fuller, S., Wiley, J., et al. (2005). A high-density SNP genomewide linkage scan for chronic lymphocytic leukemia-susceptibility loci. Am J Hum Genet 77, 420-429.
Shen, W. F., Largman, C., Lowney, P., Hack, F. M., and Lawrence, H. J. (1989). Expression of homeobox genes in human erythroleukemia cells. Adv Exp Med Biol 271, 211-219.
Stankovic, T., Stewart, G. S., Fegan, C., Biggs, P., Last, J., Byrd, P. J., Keenan, R. D., Moss, P. A., and Taylor, A. M. (2002). Ataxia telangiectasia mutated-deficient B-cell chronic lymphocytic leukemia occurs in pregerminal center cells and results in defective damage response and unrepaired chromosome damage. Blood 99, 300-309.
Stewart, C. A., Horton, R., Allcock, R. J., Ashurst, J. L., Atrazhev, A. M., Coggill, P., Dunham, Forbes, S., Halls, K., Howson, J. M., et at (2004). Complete MHC haplotype sequencing for common disease gene mapping. Genome Res 14, 1176-1187.
Stilgenbauer, S., and Dohner, H. (2005). Genotypic prognostic markers. Curr Top Microbiol Immunol 294, 147-164.
Stirzaker, C., Song, J. Z., Davidson, B., and Clark, S. J. (2004). Transcriptional gene silencing promotes DNA hypermethylation through a sequential change in chromatin modifications in cancer cells. Cancer Res 64, 3871-3877.
Tada, Y., Brena, R. M., Hackanson, B., Morrison, C., Otterson, G. A., and Plass, C. (2006). Epigenetic modulation of tumor suppressor CCAAT/enhancer binding protein alpha activity in lung cancer. J Natl Cancer Inst 98, 396-406.
Thunberg, U., Tobin, G., Johnson, A., Soderberg, 0., Padyukov, L., Hultdin, M., Klareskog, L., Enblad, G., Sundstrom, C., Roos, G., and Rosenquist, R. (2002). Polymorphism in the P2X7 receptor gene and survival in chronic lymphocytic leukaemia. Lancet 360, 1935-1939.
Toyooka, S., Toyooka, K. 0., Miyajima, K., Reddy, J. L., Toyota, M., Sathyanarayana, U. G., Padar, A., Tockman, M. S., Lam, S., Shivapurkar, N., and Gazdar, A. F. (2003). Epigenetic down-regulation of death-associated protein kinase in lung cancers. Clin Cancer Res 9, 30343041.
Wendel-Hansen, V., Sallstrom, J., De Campos-Lima, P. O., Kjellstrom, G., Sandlund, A., Siegbahn, A., Carlsson, M., Nilsson, K., and Rosen, A. (1994). Epstein-Barr virus (EBV) can immortalize B-cll cells activated by cytokines. Leukemia 8, 476-484.
Wiestner, A., Rosenwald, A., Barry, T. S., Wright, G., Davis, R. E., Henrickson, S. E., Zhao, H., Ibbotson, R. E., Orchard, J. A., Davis, Z., et al. (2003). ZAP-70 expression identifies a chronic lymphocytic leukemia subtype with unmutated immunoglobulin genes, inferior clinical outcome, and distinct gene expression profile. Blood 101, 4944-4951.
Wiley, J. S., Dao-Ung, L. P., Gu, B. J., Sluyter, R., Shemon, A. N., Li, C., Taper, J., Gallo, J., and Manoharan, A. (2002). A loss-of-function polymorphic mutation in the cytolytic P2X7 receptor gene and chronic lymphocytic leukaemia: a molecular study. Lancet 359, 1114-1119.
Yegnasubramanian, S., Kowalski, J., Gonzalgo, M. L., Zahurak, M., Piantadosi, S., Walsh, P. C., Bova, G. S., De Marzo, A. M., Isaacs, W. B., and Nelson, W. G. (2004). Hypermethylation of CpG islands in primary and metastatic human prostate cancer. Cancer Res 64, 1975-1986.
Yu, L., Liu, C., Vandeusen, J., Becknell, B., Dai, Z., Wu, Y. Z., Raval, A., Liu, T. H., Ding, W., Mao, C., et al. (2005). Global assessment of promoter methylation in a mouse model of cancer identifies ID4 as a putative tumor-suppressor gene in human leukemia. Nat Genet 37, 265-274.
Yuille, M. R., Matutes, E., Marossy, A., Hilditch, B., Catovsky, D., and Houlston, R. S. (2000). Familial chronic lymphocytic leukaemia: a survey and review of published studies. Br J Haematol 109, 794-799.

1. A method for determining susceptibility to chronic lymphocytic leukemia in a subject comprising determining a loss of expression and/or reduced expression of death associated protein kinase 1 (DAPK1), or fragments or functional equivalents thereof.

2. A method for determining susceptibility to chronic lymphocytic leukemia in a subject comprising determining a loss of expression and/or reduced expression of death associated protein kinase 1 (DAPK1), or fragments or functional equivalents thereof, in combination with frequent promoter methylation.

3. A method of claim 1, wherein, wherein determining the loss and/or reduced expression of DAPK1 includes determining a mutation in a regulatory sequence of DAPK1, by detecting at least one marker which modulates an upstream signal of DAPK1.

4. A method of claim 3, the preceding claim 3, wherein the marker comprises HOXB7.

5. A method for determining a DAPK1 genotype in a human patient, comprising:
providing a biological sample comprising nucleic acid from the patient, the nucleic acid including the patient's DAPK1 alleles;
analyzing the nucleic acid for the presence of a mutation or mutations in c.1-6531A>G of a regulatory sequence of the DAPK1 alleles, and
determining a DAPK1 genotype from the analyzing step, wherein the presence of a mutation in c.1-6531A>G is correlated with chronic lymphocytic leukemia.

6. The method of claim 5, wherein the biological sample is a cell sample.

7. The method of claim 6, wherein the cell sample comprises bone marrow or peripheral blood.

8. The method of claim 7, including a screening step wherein chronic lymphocytic leukemia (CLL) is correlated with a loss of expression and/or reduced expression of death associated protein kinase 1 (DAPK1), or fragments or functional equivalents thereof.

9. A composition comprising a single-nucleotide germline mutation (c.1-6531A>G) upstream of DAPK1 [SEQ ID NO: 1], which segregates with a CLL phenotype.

10. An isolated and purified DNA molecule having a sequence as shown in [SEQ ID NO: 1] having a mutation 6531A>G upstream of DAPK1.

11. A tumor suppressor for chronic lymphocytic leukemia (CLL) comprising one or more epigenetic silencing and/or mutations in death associated protein kinase 1 (DAPK1), or fragments or functional equivalents thereof.

12. A DAPK1 promoter construct #2, or fragments or functional equivalents thereof (c.1-1545 to c.1-1151bp) [SEQ ID NO: 2], covering bisulfite region A1 which displays extensive promoter methylation.

13. An isolated and purified DNA molecule having a sequence as shown in [SEQ ID NO: 2].

14. A chronic lymphocytic leukemia (CLL) specific SNP, comprising c.1-6531A>G in a promoter region of DAPK1.

15. A 357 by fragment including c.1-6531A (DAP-A) ligated upstream into luciferase construct #1 [SEQ ID NO: 3] containing a DAPK1 promoter.

16. An isolated and purified DNA molecule having a sequence as shown in [SEQ ID NO: 3].

17. A 357 by fragment including either c.16531G (DAP-G) ligated upstream into luciferase construct #1 [SEQ ID NO: 4] containing a DAPK1 promoter.

18. An isolated and purified DNA molecule having a sequence as shown in [SEQ ID NO: 4].

19-52. (canceled)


SEQUENCE LISTING (9 sequences)

SEQ ID NO: 1 - DNA, 36 bp, Artificial Sequence
  Description of Artificial Sequence Synthetic oligonucleotide
cttgccttggtcgtggttacctacagatgcctgaat

SEQ ID NO: 2 - DNA, 395 bp, Artificial Sequence
  Description of Artificial Sequence Synthetic construct
ggacggaagaaccgtgtttccctagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgc

SEQ ID NO: 3 - DNA, 357 bp, Artificial Sequence
  Description of Artificial Sequence Synthetic construct
caggtcctgtgatttctacttccttaaagtatctcattagttttctatacccaccatctgtgctaaattagtattaattaactatctttagagtgatacagccacatgctgagcatgatggacaaggaccttcaagctctaccttggttcacccctccatctcatctgagttctgggcatgatggaagagactcttctcctctccactcttgcctcttgccattaccttgccttggtcgtgattacctacagatgcctgaatgagtcatagtctttctcatatccaggattgtacccttgctgttttcctctgcctagatgccctcctttccaacccccactgtccaataacccacc

SEQ ID NO: 4 - DNA, 357 bp, Artificial Sequence
  Description of Artificial Sequence Synthetic construct
caggtcctgtgatttctacttccttaaagtatctcattagttttctatacccaccatctgtgctaaattagtattaattaactatctttagagtgatacagccacatgctgagcatgatggacaaggaccttcaagctctaccttggttcacccctccatctcatctgagttctgggcatgatggaagagactcttctcctctccactcttgcctcttgccattaccttgccttggtcgtggttacctacagatgcctgaatgagtcatagtctttctcatatccaggattgtacccttgctgttttcctctgcctagatgccctcctttccaacccccactgtccaataacccacc

SEQ ID NO: 5 - DNA, 36 bp, Artificial Sequence
  Description of Artificial Sequence Synthetic oligonucleotide
cttgccttggtcgtgattacctacagatgcctgaat

SEQ ID NO: 6 - DNA, 36 bp, Artificial Sequence
  Description of Artificial Sequence Synthetic oligonucleotide
cttgccttggtcgtaactacctacagatgcctgaat

SEQ ID NO: 7 - DNA, 36 bp, Artificial Sequence; identical to SEQ ID NO: 1
  Description of Artificial Sequence Synthetic oligonucleotide

SEQ ID NO: 8 - DNA, 36 bp, Artificial Sequence
  Description of Artificial Sequence Synthetic oligonucleotide
cttgccttggtcgtagctacctacagatgcctgaat

SEQ ID NO: 9 - DNA, 1065 bp, Artificial Sequence
  Description of Artificial Sequence Synthetic construct
tcacttgaaccctggaggtggaggttgccacgctccactacactccagcctggcgacagagtgagactccgtctcaaaaaaaagaaagtaaacaggtgaaattaattttaataatatattttgtttaacccaacgtatccaaaatactatcatttgaaagtgtaatgaatataaaaatattcatgagatatttttcattctcatatccatactgtcttggactctaatgtgtattttacacttacagcacaattaatttgggactagctacatttcagctcaacaatagccaatagcatatgggatagcgcaaataaactctgcgtctctgttgcttctttgggtctcggagacctcaaccctttcttcagattgcaaaccttcttgccttcaagcctcggctccaacaccagtccggcagaggaacccagtctaatgaggtacgctcccttcctgccattctctattccattaacctgtttcgtggtaaacgtaggactgatcctccaaaattaccttattaattagcttacatatttattatctatctgtcccaccagaatgcaggtttccggaaggcagggatttaaaaaaatctgttttgttctatgtgattttcccataccaagcaccgtgcccggcacaagctgggatcccagtacacatctcgggacggaagaaccgtgtttccctagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgc